(19) United States
(12) Patent Application Publication
(10) Pub. No.: US 2025/0114368 A1
(43) Pub. Date: Apr. 10, 2025

(54) COMPOSITIONS AND METHODS RELATING TO PEDIATRIC NEUROMUSCULAR DISEASES AND NEUROGENERATIVE DISORDERS

(71) Applicant: Ann and Robert H. Lurie Children's Hospital of Chicago, Chicago, IL (US)

(72) Inventor: Yongchao C. MA, Chicago, IL (US)

(21) Appl. No.: 18/908,483

(22) Filed: Oct. 7, 2024

Related U.S. Application Data
(60) Provisional application No. 63/588,222, filed on Oct. 5, 2023.

(51) Int. Cl. A61K 31/52; A61K 31/155; A61K 31/16; A61K 31/17; A61K 31/192; A61K 31/216; A61K 31/27; A61K 31/336; A61K 31/4015; A61K 31/415; A61K 31/426; A61K 31/4439; A61K 31/506; A61K 31/519; A61K 38/06; A61K 38/17; A61P 25/28; C12N 15/113

(52) U.S. Cl. CPC A61K 31/52; A61K 31/155; A61K 31/16; A61K 31/17; A61K 31/192; A61K 31/216; A61K 31/27; A61K 31/336; A61K 31/4015; A61K 31/415; A61K 31/426; A61K 31/4439; A61K 31/506; A61K 31/519; A61K 38/06; A61K 38/1709; A61P 25/28; C12N 15/1137; C12N 2310/11; C12N 2310/12; C12N 2310/141; C12N 2310/3233; C12N 2310/531

(57) ABSTRACT

The present disclosure provides compositions and methods related to pediatric neuromuscular diseases and neurodegenerative disorder. In particular, the present disclosure provides compositions and methods for treating and/or preventing spinal muscular atrophy (SMA) and neurodegenerative disorders with dysregulation of Cdk5 signaling and mitochondrial defects.

### CROSS REFERENCE TO RELATED APPLICATION

The present application claims priority to U.S. Provisional application Ser. No. 63/588,222, filed Oct. 5, 2023, which is herein incorporated by reference in its entirety.

### STATEMENT REGARDING FEDERALLY SPONSORED RESEARCH OR DEVELOPMENT

This invention was made with government support under grant numbers R21NS106307 and R01NS094564 awarded by the National Institutes of Health. The government has certain rights in the invention.

### FIELD

The present disclosure provides compositions and methods related to pediatric neuromuscular diseases and neurodegenerative disorder. In particular, the present disclosure provides compositions and methods for treating and/or preventing spinal muscular atrophy (SMA) and neurodegenerative disorders with dysregulation of Cdk5 signaling and mitochondrial defects.

### BACKGROUND

Spinal muscular atrophy (SMA) is a fatal pediatric neuromuscular disease that affects approximately 1 in 6000 live births in the United States, ranking as the number one genetic cause of infant mortality. There is currently no consensus on the mechanisms underlying the onset (e.g., motor neuron degeneration) of spinal muscular atrophy (SMA). Compositions and methods to elucidate such mechanisms are needed.

### SUMMARY

Embodiments of the present disclosure include compositions that include a Cdk5 inhibitor (e.g., BML259) or a calpain inhibitor (e.g., Calpeptin).

Embodiments of the present disclosure also include methods of treating a subject who suffers from a pediatric neuromuscular disease (e.g., spinal muscular atrophy (SMA)) comprising administering any of the compositions described herein to a subject in need thereof. In some embodiments, the pediatric neuromuscular disease is SMA.

Embodiments of the present disclosure also include methods of treating a subject who suffers from a neurodegenerative disorder (e.g., neurodegenerative disorders with dysregulation of Cdk5 signaling and/or mitochondrial defects) comprising administering any of the compositions described above to a subject in need thereof.

In some embodiments, the composition is administered orally, parenterally, intramuscularly, intraperitoneally, intravenously, intracerebroventricularly, intracisternally, intratracheally, intranasally, subcutaneously, via injection or infusion, via inhalation, spray, nasal, vaginal, rectal, sublingual, or topical administration. In some embodiments, the composition is administered via nebulization to lung tissue.

Embodiments of the present disclosure also include methods of treating a subject who suffers from a pediatric neuromuscular disease (e.g., SMA) comprising targeting the Cdk5 signaling pathway. In some embodiments, the method includes inhibiting Cdk5 activation. In some embodiments, the method includes improving mitochondrial function (e.g., rescuing mitochondrial transport and fragmentation; reducing mitochondrial oxidative stress; aberrant Cdk5 activation) by inhibiting the expression or activity of p35.

Embodiments of the present disclosure also include methods of treating a subject who suffers from other neurodegenerative disorders (e.g., neurodegenerative disorders with dysregulation of Cdk5 signaling and/or mitochondrial defects) comprising targeting the Cdk5 signaling pathway. In some embodiments, the method includes inhibiting Cdk5 activation. In some embodiments, the method includes reducing Cdk5 activation. In some embodiments, the method includes improving mitochondrial function (e.g., rescuing mitochondrial transport and fragmentation; reducing mitochondrial oxidative stress; aberrant Cdk5 activation) by inhibiting the expression or activity of p35.

Embodiments of the present disclosure also include methods of treating or preventing a pediatric neuromuscular disease comprising administering a Cdk5 inhibitor or calpain inhibitor to a subject suffering from or at risk of a pediatric neuromuscular disease.

Embodiments of the present disclosure also include methods of treating or preventing a neurodegenerative disorder comprising administering a Cdk5 inhibitor or calpain inhibitor to a subject suffering from or at risk of a neurodegenerative disorder.

In some embodiments, the pediatric neuromuscular disease or the neurodegenerative disorder is spinal muscular atrophy.

In some embodiments, the neurodegenerative disorder is spinal muscular atrophy.

In some embodiments, the neurodegenerative disorder is a neurodegenerative disorder with dysregulation of Cdk5 signaling and mitochondrial defects.

Embodiments of the present disclosure also include a method of treating a subject who suffers from a pediatric neuromuscular disease comprising targeting the Cdk5 signaling pathway.

Embodiments of the present disclosure also include a method of treating a subject who suffers from a pediatric neuromuscular disease comprising inhibiting Cdk5 activation.

Embodiments of the present disclosure also include a method of treating a subject who suffers from a pediatric neuromuscular disease comprising reducing Cdk5 activation.

Embodiments of the present disclosure also include a method of treating a subject who suffers from a pediatric neuromuscular disease comprising improving mitochondrial function by genetically knocking out p35 (or otherwise inactivating, inhibiting, or reducing the expression of p35).

In some embodiments, improving mitochondrial function comprises rescuing mitochondrial transport and fragmentation.

In some embodiments, improving mitochondrial function comprises reducing mitochondrial oxidative stress.

In some embodiments, improving mitochondrial function comprises reducing aberrant Cdk5 activation.

Embodiments of the present disclosure also include a method of treating a subject who suffers from a neurodegenerative disorder comprising targeting the Cdk5 signaling pathway.

Embodiments of the present disclosure also include a method of treating a subject who suffers from a neurodegenerative disorder comprising inhibiting Cdk5 activation.

Embodiments of the present disclosure also include a method of treating a subject who suffers from a neurodegenerative disorder comprising reducing Cdk5 activation.

Embodiments of the present disclosure also include a method of treating a subject who suffers from a neurodegenerative disorder comprising improving mitochondrial function by genetically knocking out p35 (or otherwise inactivating, inhibiting, or reducing the expression of p35).

In some embodiments, improving mitochondrial function comprises rescuing mitochondrial transport and fragmentation.

In some embodiments, improving mitochondrial function comprises reducing mitochondrial oxidative stress.

In some embodiments, improving mitochondrial function comprises reducing aberrant Cdk5 activation.

In some embodiments, the Cdk5 inhibitor comprises a BML-259, a CDK5-IN-4, a 25-106, a seliciclib, a roscovitine, a CY-202, a quinazolinone derivative, a quercetin, a dinaciclib, CDK5 inhibitory peptide (CIP), a derivative of a P5 peptide (e.g., conjugated with a cell-penetrating transactivator of transcription (TAT) sequence), a statin, a metformin, a fenofibrate, and a rosiglitazone.

In some embodiments, the calpain inhibitor comprises calpeptin, N-acetyl-leu-leu-norleucinal (ALLN), PD150606, SNJ-1945, AK275, MDL28170, Gabadur, Neurodur, E-64-d, SCI Leupeptin, and NA-184.

In some embodiments, the administering comprises co-administration of metformin and fenofibrate, co-administration of metformin and rosiglitazone, and co-administration of rosiglitazone and glimepiride, and/or other combinations of the Cdk5 inhibitors and/or calpain inhibitors described herein.

In some embodiments, the Cdk5 inhibitor and/or calpain inhibitor is formulated in any suitable manner for the route of administration, such as a tablet, a capsule, infusion, a liquid, an aerosol, etc.

In some embodiments, the Cdk5 inhibitor and/or calpain inhibitor is formulated with any additional components suitable for the route of administration, such as a disintegrant, a super disintegrant, and a sugar-based excipient, etc.

In some embodiments, the administering comprises oral, parenteral, intramuscular, intraperitoneal, intravenous, intracerebroventricular, intracisternal, intratracheal, intranasal, subcutaneous, via injection or infusion, via inhalation, spray, nasal, vaginal, rectal, sublingual, and topical administration.

### BRIEF DESCRIPTION OF THE DRAWINGS

FIGS. 1A-1H: Endogenous Cdk5 kinase activity is upregulated in SMA mouse spinal cords before the onset of disease symptoms. (A-B) Cdk5 kinase activity is significantly elevated in spinal cords from Δ7 SMA mice (Smn^{-/-}; SMN^{tg/tg}; SMNAΔ7^{tg/tg}) both at postnatal day 3 (P3) and postnatal day 9 (P9). Endogenous Cdk5 was immunoprecipitated from spinal cord lysates of P3 and P9 mice. Kinase activity of immunoprecipitated Cdk5 was measured by its phosphorylation of Histone H1 with ³²P-γ-ATP, shown by autoradiography in (A). The same levels of Cdk5 and purified Histone H1 used in kinase assay reactions were monitored by Western blotting. Kinase activity was quantified (B) using a phosphor imager and scintillation counter. Data are from four independent experiments and are mean±SEM. ** P<0.01, Paired Student's t test. WT: wild type; SMA: spinal muscular atrophy. (C-D) Time course of motor symptom onset in SMA mice. From P5-P9, A7 SMA mice (blue line, n=21) took significantly longer to right themselves compared to wild type control littermates (magenta line, n=18) (C). By P7 significantly more wild type control littermates (magenta line, n=18) could successfully climb 550 incline in one minute compared to SMA mice (blue line, n=21) (D). (E-F) Relative size of SMA mice at P3 and P9. At P3 (E) A7 SMA mice and wild type control mice are very similar in size, while at P9 (F) SMA mice are significantly smaller and less active. (G-H) Cdk5 kinase activity is also upregulated in the Hung-Li SMA mouse model (Smn^{+/-}; SMN2Hung^{tg/tg}). Endogenous Cdk5 was immunoprecipitated from P3 and P9 spinal cord lysates, and the kinase activity was measured by its phosphorylation of Histone H1 with ³²P-γ-ATP. Data are from four independent experiments and are mean±SEM. ** P<0.01, Paired Student's t test.

FIGS. 2A-2B: Increased conversion of p35 to p25 in SMA mouse spinal cords. (A) Western blot analysis shows that p25 protein level is significantly increased at both pre-symptomatic (P3) and post-symptomatic (P9) stages in spinal cords from Δ7 SMA mice. Neither p35 nor p25 was detected in p35 knockout mice (p35^{-/-}). Expression levels of GAPDH detected by Western blotting were used as control. (B) Quantification of the p35 to p25 ratio in Δ7 SMA mice by measuring integrated optical density. Data are from four independent experiments and are mean±SEM. ** P<0.01, Student's t test.

FIGS. 3A-3J: Upregulation of Cdk5 activity and p25 level are conserved in human iPSC models of SMA. (A-F) Human iPSCs and iPSC-derived motor neurons serve as a model for studying SMA. Brightfield microscopy (A) and immunofluorescence staining (B-F) identify iPSCs (A-B) generated from SMA patient skin fibroblasts expressing the pluripotency marker Oct4 (green in B), and iPSC-derived neural progenitors expressing Nestin (green in C). After six weeks in culture human iPSC-derived motor neurons (D-F) express both neuronal marker TuJ1 (green in D, F) and motor neuron marker Isl1 (red in D, F). Cell nuclei were stained with DAPI (blue in E and F). (G-J) Endogenous Cdk5 kinase activity and the conversion of p35 to p25 are increased in human SMA iPSC-derived motor neurons. Cdk5 was immunoprecipitated from WT and SMA human iPSC-derived motor neurons after six weeks in culture. Kinase activity (G) was measured by its phosphorylation of Histone H1 with ³²P-γ-ATP, shown by autoradiography in (G). The level of Cdk5 in each kinase assay reaction was monitored by Western blot. Kinase activities were quantified (I) using a phosphor imager. The ratio of p25 to p35 was measured by Western blot analysis (H) and quantified by densitometry (J) using GAPDH as equal loading control. Images are representative from four experiments and data are mean±SEM. ** P<0.01, Paired t test for (I) and unpaired t test for (J).

FIGS. 4A-4D: Mitigating aberrant Cdk5 activation ameliorates the degeneration of motor neurons from SMA mice. (A-B) Primary spinal motor neurons from SMA mice cultured 6 days in vitro (6 DIV) were stained by antibodies recognizing motor neuron marker Isl1 (magenta in A, B) and pan-neuronal marker TuJ1 (green in A). DAPI nuclear staining marks all cells in the culture. (C) Western blot analysis of Cdk5 knockdown in motor neurons treated with shRNA. Lentivirus-mediated shRNA targeting Cdk5, but not the scrambled control shRNA, can effectively knockdown the expression of Cdk5, but not GSK3β, in primary mouse spinal motor neurons. Motor neurons were infected with lentivirus on 4 DIV and cultured for another 3 days before Western blot analysis. Two examples of Cdk5 shRNA knockdown were shown, with the expression of GAPDH as loading control. (D) Quantification of TUNEL+ degenerating motor neurons from WT and SMA mice. Inhibition of Cdk5 activity by applying 300 nM Cdk5 inhibitor BML259 or lentiviral shRNA to knockdown Cdk5 in cultured motor neurons at 3 DIV significantly reduced the degeneration of SMA motor neurons assayed on 6 DIV. Data are eight sets from four independent experiments and are mean±SEM. ** P<0.01, *** p<0.001, Student's t test.

FIGS. 5A-5J: Genetic knockout of p35 rescues mitochondrial defects in SMA mouse motor neurons. (A-D) Representative kymographs of mitochondrial axon transport in wild type, SMA, SMA;p35^{-/-} and p35^{-/-} mice primary motor neurons. (E-H) Quantification of mitochondrial transport. The percentage of mobile mitochondria that travel more than 1 μm per minute (E) and the average total travel distance of all tracked mitochondria (F) are significantly reduced in SMA motor neurons, and rescued to the wild type level by genetic knockout of p35 in SMA mice. The average total anterograde travel distance of all analyzed mitochondria is not change in SMA motor neurons, while the retrograde travel distance is significantly reduced (H), which is restored to the wild type level by the knockout of p35 in SMA motor neurons. Quantification in E-H is from 27 neurons from each genotype (WT: n=259; SMA: n=236; SMA;p35^{-/-}: n=273; p35^{-/-}: n=181 mitochondria) and are mean±SEM. *** P<0.001, one-way ANOVA with Tukey HSD post hoc analysis. (I-J) Genetic knockout of p35 rescues mitochondrial fragmentation in SMA motor neurons. (I) Representative images for measuring mitochondrial length in wild type, SMA, SMA;p35^{-/-} and p35^{-/-} mouse motor neurons transfected with mito-dsRed and quantified using confocal live imaging. (J) Mitochondrial length in spinal motor neurons from SMA mice is significantly shorter, which is rescued to the wild type level by the knockout of p35 in SMA mice. Quantification in J is from 27 neurons from each genotype (WT: n=211; SMA: n=228; SMA;p35^{-/-}: n=187; p35^{-/-}: n=122 mitochondria), and are mean±SEM. **** P<0.0001, one-way ANOVA with Tukey HSD post hoc analysis.

FIGS. 6A-6H: Genetic knockout of p35 alleviates synaptic defects and hyperexcitability in SMA mouse motor neurons. (A-E) Excitatory glutamatergic synapse loss on SMA spinal cord motor neurons can be rescued by p35 knockout. Colocalization of the glutamatergic synapse marker vesicular glutamate transporter 1(vGluT1) (green in A-D) with the motor neuron marker choline acetyltransferase (ChAT) (red in A-D) by immunostaining, shows that excitatory synaptic boutons on motor neurons are significantly reduced in SMA mice, and rescued to the wild type level by genetic knockout of p35 in SMA mice (E). Quantification in E is from three to four mice from each genotype (WT: n=42; SMA: n=51; SMA;p35^{-/-}: n=54; p35^{-/-}: n=34 motor neurons) and are mean±SEM. *** P<0.001, one-way ANOVA with Tukey HSD post hoc analysis. (F-H) SMA spinal motor neuron hyperexcitability can be reduced by genetic knockout of p35. Example plot of a firing motor neuron recorded during depolarizing current ramps (F). Threshold voltage (green), I-ON (red) and I-OFF (blue) were measured as shown. (G-H). Whole cell patch clamp electrophysiological recording of spinal motor neurons from P7-9 SMA mice showed that the amplitude (G) of persistent inward current (PIC), which indicates the level of cellular excitability, was significantly increased in SMA motor neurons, and rescued in SMA;p35^{-/-} mice. Threshold voltage (H) of action potential (AP) firing is significantly more hyperpolarized in SMA motor neurons, but not in motor neurons from SMA;p35^{-/-} mice. Quantifications are from four mice of each genotype and are mean±SEM. * P<0.05, ** P<0.01, one-way ANOVA with Tukey HSD post hoc analysis.

FIGS. 7A-7I: Genetic knockout of p35 rescues neuromuscular junction defects and motor neuron degeneration in SMA mice. (A-E) Motor neuron axon denervation at NMJs in SMA mice can be rescued by genetic knockout of p35. Co-localization of presynaptic nerve terminals marked by immunostaining with anti-neurofilament (NF) and anti-SV2 antibodies (green in A-D) with post-synaptic acetylcholine receptors labeled by α-bungarotoxin (αBTX) (red in A-D) shows increased denervation (arrow heads) and partial innervation (arrow) of NMJs on the FDB-2 muscle of SMA mice. The NMJ innervation defect is rescued to the wild type level in SMA;p35^{-/-} mice. Quantification in E is from over 120 NMJs on the FDB-2 muscle from each mouse. Five mice for each genotype were used. Denervated, partially innervated and fully innervated NMJs were analyzed separately. Results are mean±SEM. *** P<0.001, one-way ANOVA with Tukey HSD post hoc analysis. (F-J) Motor neuron degeneration in SMA mice is reduced by p35 knockout. Lumbar level spinal cord sections of P9 SMA mice show a near 40% reduction of Islet1-positive (red in F-I) motor neurons compared to wild type littermates. Genetic knockout of p35 reduces motor neuron degeneration in SMA;p35^{-/-} mice (J). Quantification in J is from four to five mice from each genotype in four independent experiments (WT: n=130; SMA: n=144; SMA;p35^{-/-}: n=162; p35′: n=129 sections) and are mean±SEM. *** P<0.001, one-way ANOVA with Tukey HSD post hoc analysis.

FIGS. 8A-8M: Inhibition of Cdk5 signaling pathway decreases the degeneration of human SMA iPSC-derived motor neurons. (A-L) Immunostaining of human iPSC-derived motor neurons with antibodies recognizing neuronal marker TuJ1 (green in A-L), motor neuron marker Isl1 (red in A-L), and nucleus marker DAPI (blue in A-L). Treatment of human SMA iPSC motor neurons from week 3 to 6 in culture with a calpain inhibitor significantly increases the number of Isl1-positive motor neurons, while DMSO vehicle control has no effect. (M) Quantification of TuJ1 and Isl1-positive motor neurons as a percentage of all DAPI positive cells in a human iPSC model of SMA treated with inhibitors of Cdk5 signaling. Application of 300 nM Cdk5 inhibitor BML259 or 10 μM calpain inhibitors Calpeptin from week 3 to week 6 in human SMA iPSC culture significantly reduces the degeneration of motor neurons. Data are from four independent experiments and are mean±SEM. ** P<0.01, *** p<0.001, **** P<0.0001, Student's t test.

FIGS. 9A-C: (A) The increase of Cdk5 kinase activity in E18.5 SMA mouse spinal cord is not significant. Endogenous Cdk5 was immunoprecipitated from spinal cord lysates of E18.5 WT and SMA mice and measured kinase activity of phosphorylating Histone H1 with ³²P-γ-ATP. The same levels of Cdk5 and purified Histone H1 used in kinase assay reactions were monitored by Western blotting. Data are mean±SEM, Student's t test (n=12, P=0.0875). (B) Cdk5 kinase activity is significantly increased in a SMA iPSC model created by non-integrating episomal reprogramming. Endogenous Cdk5 was immunoprecipitated from motor neurons differentiated from WT and SMA non-integrating iPSCs. Kinase activities were quantified by the phosphorylation of Histone H1 with ³²P-γ-ATP. The same levels of Cdk5 and Histone H1 were monitored by Western blot. Data are mean±SEM, Student's t test (n=12, P<0.0001). (C) Inhibition of the Cdk5 signaling pathway significantly reduces motor neuron degeneration in non-integrating iPSC SMA model. Calpain inhibitor Calpeptin (10 μM) was applied to WT and SMA non-integrating iPSC-derived motor neurons. The number of Isl1+ and TuJ1+motor neurons as a percentage of all DAPI-positive cells was quantified from 1490 motor neurons in four independent experiments and are mean±SEM. ** p=0.0045, Student's t test.

FIGS. 10A-C: (A-B) Inhibition of Cdk5 signaling reduces iPSC motor neuron degeneration measured by staining with motor neuron markers choline acetyltransferase (ChAT) (A) or Terminal Transferase dUTP Nick End Labeling (TUNEL) (B). Calpeptin (10 μM) (A) was used to inhibit calpain activity, and 300 nM BML259 (B) was used to inhibit Cdk5 activity in control and SMA iPSC motor neurons. Data are from four independent experiments and are mean±SEM. ** p<0.01, *** p<0.001, Student's t test. (C) RT-qPCR measurement of mRNA expression shows significantly decreased bdnf level in spinal cords from P3 Δ7 SMA mice compared to WT control littermates. Levels of different mRNA in each sample are calculated as relative to gapdh mRNA. The level of gapdh mRNA is presented as relative to the average of four samples. The measurement of each sample was repeated three times and four sets of mice were used. Results are mean±SEM. ** p<0.01, Student's t test.

FIGS. 11A-B: (A) Endogenous Cdk5 kinase activity in post-symptomatic postnatal natal day 9 (P9) spinal cords of Hung-Li SMA mice is significantly increased compared to WT, SMA;p35^{-/-}, and p35^{-/-} mice (****, P<0.0001, one-way ANOVA). Tukey's multiple comparisons test shows the difference among WT, SMA;p35^{-/-}, and p35^{-/-} mice are not significant. (B) Cdk5 activity in pre-symptomatic postnatal natal day 3 (P3) spinal cords of Hung-Li SMA mice is significantly increased compared to WT, SMA;p35^{-/-}, and p35^{-/-} mice (****, P<0.0001, one-way ANOVA). Tukey's multiple comparisons test shows the difference among WT, SMA;p35^{-/-}, and p35^{-/-} mice are not significant, while the adjusted P value for WT vs. SMA is 0.002 (**), for SMA vs. SMA;p35^{-/-} is <0.0001 (****), and for SMA vs. p35^{-/-} is <0.0001 (****). Endogenous Cdk5 was immunoprecipitated from mouse spinal cord lysates and measured kinase activity of phosphorylating Histone H1 with ³²P-γ-ATP. Data are from four independent experiments and are mean±SEM.

### DETAILED DESCRIPTION

Embodiments of present disclosure provide compositions and methods related to pediatric neuromuscular diseases. In particular, the present disclosure provides compositions and methods for treating and/or preventing spinal muscular atrophy (SMA).

Spinal muscular atrophy (SMA) is a fatal pediatric neuromuscular disease that affects approximately 1 in 6000 live births in the United States, ranking as the number one genetic cause of infant mortality. SMA is characterized by the degeneration of motor neurons (i.e., nerve cells that control muscle movement) causing progressive wasting and paralysis of muscles, respiratory distress, and, ultimately, death. There is currently no consensus on the mechanisms underlying the onset (e.g., motor neuron degeneration) of spinal muscular atrophy (SMA).

Experiments conduscted during development of embodiments herein demonstrate that the activity of cyclin-dependent kinase 5 (Cdk5) is upregulated in mouse models and human induced pluripotent stem cell (iPSC) models of SMA. The increase of Cdk5 activity occurs before the onset of SMA phenotypes, indicating that it is an initiator of the disease. Aberrant Cdk5 activation causes mitochondrial defects and motor neuron degeneration. Inhibition of the Cdk5 signaling pathway by small molecule inhibitors and genetic reduction of Cdk5 activity reduces the degeneration of motor neurons derived from SMA mice and human SMA iPSCs, rescues mitochondrial defects, and alleviates SMA disease phenotypes. Altogether, the embodiments of present disclosure reveal a critical role for the aberrant activation of Cdk5 in SMA pathogenesis and indicate a target for therapeutic intervention.

Using mouse models and human iPSC models of SMA, it was revealed that Cdk5 activity is significantly increased in SMA pathogenesis before the onset of disease symptoms. Genetic reduction of Cdk5 activation in SMA mice rescues mitochondrial defects and other diverse SMA disease phenotypes in vivo. Notably, inhibition of Cdk5 signaling ameliorates the degeneration of motor neurons derived from human SMA iPSCs and SMA mouse models, providing new insights into the pathogenic mechanism and a therapeutic strategy for SMA.

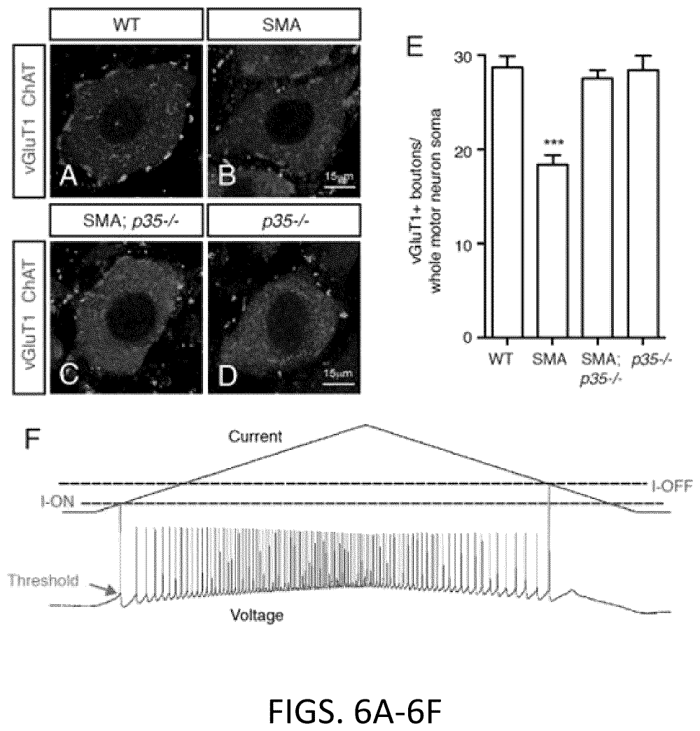

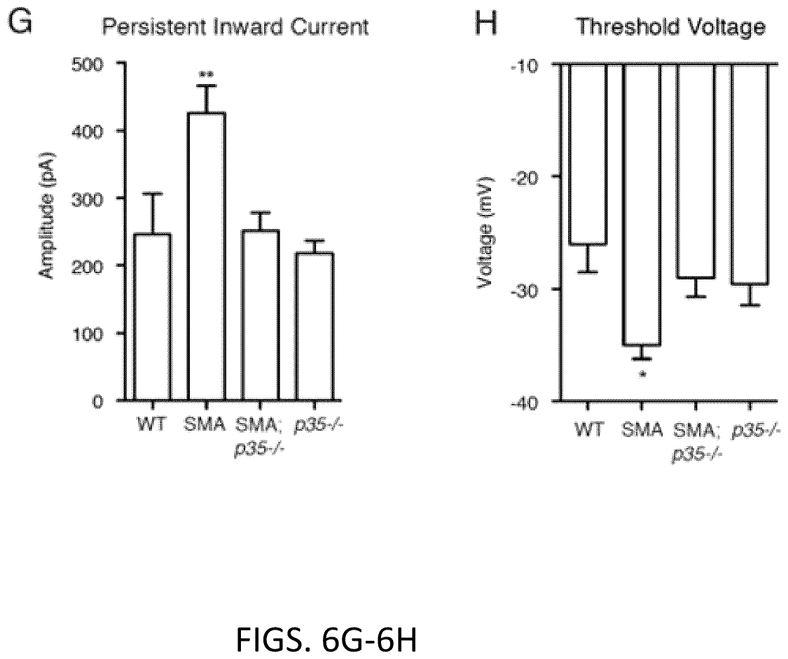

A Positive Feedback Loop Formed by Aberrant Cdk5 Activation and Mitochondrial Defects leading to Motor Neuron Degeneration in SMA. One finding from the experiments conducted during development of embodiments herein is that genetic knockout of p35 rescues mitochondrial defects in motor neurons affected by SMA, revealing a critical signaling mechanism underlying the mitochondrial phenotypes in the identified SMA motor neurons. Mitochondria play a central role in generating energy and buffering Ca²⁺. In highly polarized motor neurons, healthy mitochondria are dynamically transported to regions that need more energy and Ca²⁺ buffering. Mitochondrial functions are closely affected by its size and morphology. Elongated mitochondria have higher density of cristae with better efficiency in ATP production; whereas short and fragmented mitochondria, as seen in SMA motor neurons, are more likely to have compromised Ca²⁺ buffering capacity and low efficiency in ATP production. The hyperexcitability of SMA motor neurons, shown by electrophysiology recordings (FIGS. 6F-6H, Table 1), leads to their particularly high demand for energy supply and Ca²⁺ buffering. However, mitochondrial transport and fragmentation defects in SMA motor neurons reduce their capacity for energy production and Ca²⁺ buffering, further exacerbating the abnormal increase of intracellular Ca²⁺ and energy shortage created by hyperexcitability. Consequently, increased Ca²⁺ activation of calpain and upregulated calpain conversion of p35 to p25 further activate Cdk5, and Cdk5 hyperphosphorylation of targets that cause mitochondrial dysfunction leads to more severe energy shortage and disrupted Ca²⁺ buffering. Activation of this positive feedback loop in SMA pathogenesis eventually turns motor neuron hyperexcitability to excitotoxicity and degeneration. The findings highlight an aberrant Cdk5 activation-centered mechanism that contributes to SMA pathogenesis.

The Pre-symptomatic Upregulation of Cdk5 Activity in SMA. The activation of Cdk5 found in SMA is different from other neurodegenerative disorders. First, in SMA mouse models Cdk5 activity is upregulated before the onset of disease phenotypes, suggesting that increased Cdk5 activity may be a cause, rather than a consequence, of SMA pathogenesis. Significant upregulation of Cdk5 activity was observed in SMA mouse models as early as postnatal day 3 (P3), when SMA and non-disease littermate control mice are indistinguishable at the behavioral and cellular levels, suggesting that aberrant Cdk5 activation may be part of the mechanisms that initiate SMA disease. Thus, mitigating Cdk5 activity represents a strategy to alleviate SMA pathology through early intervention. Second, the upstream cause of Cdk5 hyperactivation in SMA, a pediatric neuromuscular disorder, is likely different from that in aging-associated neurodegeneration. Both genetic mutations and age-dependent non-genetic risk factors contribute to the pathogenesis of Alzheimer's disease and ALS. By contrast, SMA is a monogenic disorder caused by mutations in SMN1 gene, leading to reduced SMN protein level. SMN has been implicated in regulating snRNP biogenesis and pre-mRNA splicing. RNA splicing and expression defects in calcium channel and other mechanisms regulating calcium homeostasis have been revealed by RNA-Seq analysis of motor neurons affected by SMA. These defects indicate a link that connects reduced SMN protein level, through disrupting calcium homeostasis and increased Ca²⁺-dependent calpain cleavage of p35 to p25, with aberrant Cdk5 activation in SMA

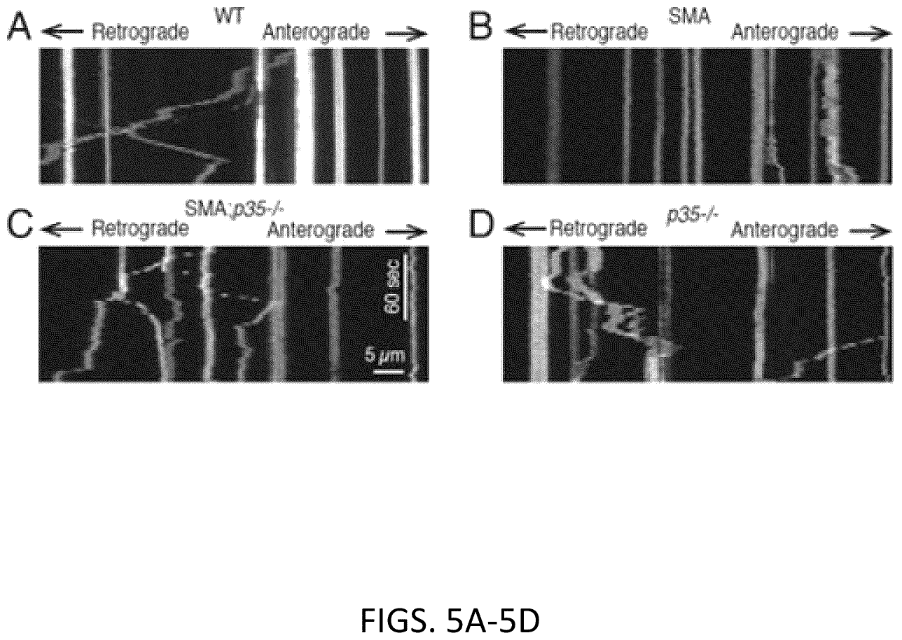

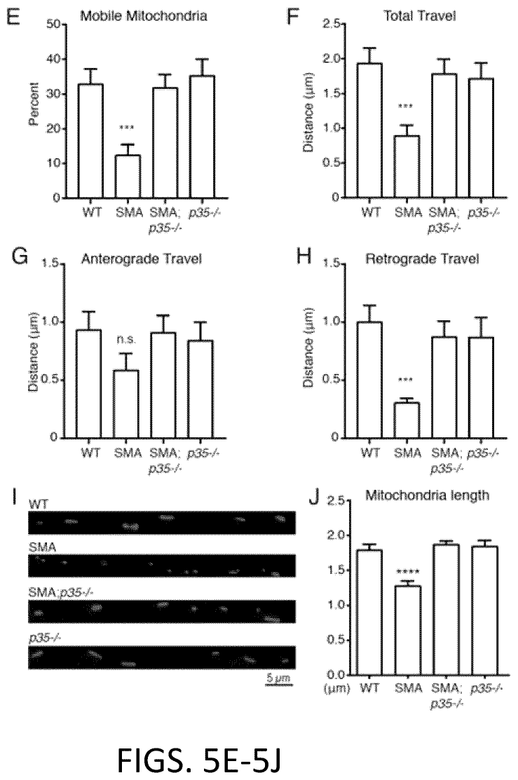

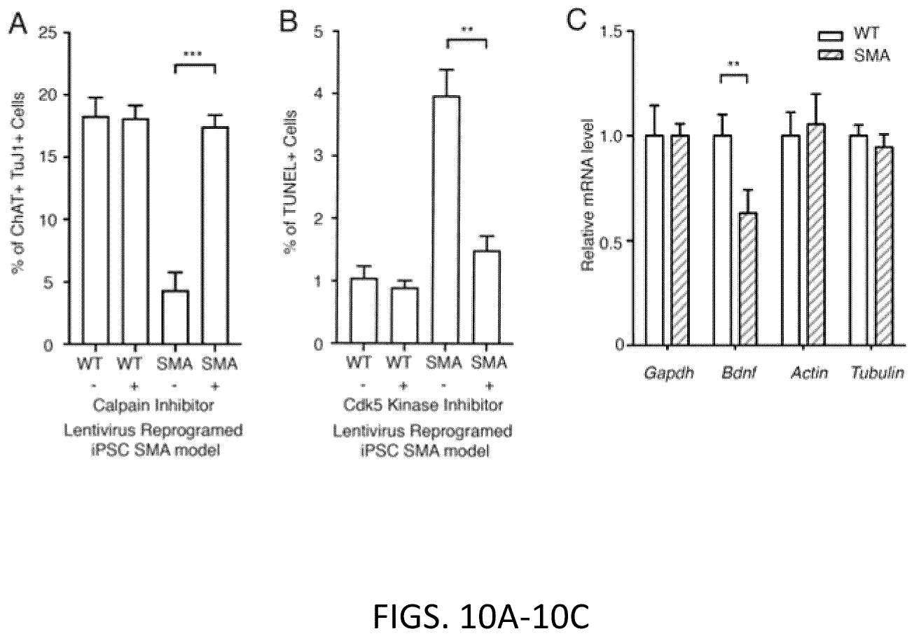

Cdk5 Targets in SMA Pathogenesis. MDAS domain transcription factor myocyte enhancer factor 2 (MEF2), dynein-interacting nuclear distribution protein nudE-like 1 (Ndel1), mitochondrial fission regulator dynein related protein 1 (Drp1), and microtubule-associated protein tau, which can be phosphorylated by Cdk5, are downstream targets of Cdk5. It was found that microtubule-associating protein tau phosphorylation by Cdk5 was dramatically increased in SMA without causing protein fibrillary tangles or aggregates, which is different from neurodegenerative disorders associated with upregulated Cdk5 activity and protein aggregation. Experiments conducted during development of embodiments herein indicate that in SMA, increased Cdk5 phosphorylation of tau contributes to disease pathogenesis by disrupting axonal transport and tau-interaction with prosurvival factors, and by causing excitotoxicity. Increased phosphorylation of tau impairs microtubule assembly in axons, and compromises microtubule-dependent protein and organelle transport, which is particularly exacerbated in spinal motor neurons that have long axons, contributing to the selective vulnerability of motor neurons in SMA. In addition, experiments conducted during development of embodiments herein indicate that increased tau phosphorylation in SMA disrupt complexes formed by tau-binding partners, including phosphatidylinositol 3-kinase (PI3K) and Src family kinases that are critical for neuronal survival. Moreover, tau has been shown to be required for targeting Src family kinase Fyn to phosphorylate NR2B and prevent NMDA receptor-mediated excitotoxicity. Experiments indicate that increased phosphorylation of tau disrupts the balance between excitatory and inhibitory synaptic inputs on motor neurons, leading to excitotoxicity in SMA. Regarding MEF2, it regulates neuronal survival, neurite outgrowth, and synapse elimination, which are very relevant to the central excitatory synapse loss phenotype and NMJ denervation observed in SMA. The temporal expression of MEF2 peaks at the early postnatal stage when synapse elimination and motor neuron target innervation is most active. One critical MEF2 target gene involved in regulating axonal morphogenesis, synaptic elimination and neuronal survival is brain derived neurotrophic factor (bdnf). When the expression of bdnf in SMA mouse spinal cords using RT-qPCR (quantitative reverse transcription PCR) is measured, bdnf expression showed significant decrease in spinal cords from SMA mice compared to control littermates (FIG. 10D), supporting a role for the dysregulation of MEF2 target gene expression in SMA pathogenesis. In addition, MEF2 has been shown to be physically present in mitochondria and function as a mitochondrial transcription factor to directly regulate the expression of mitochondrial complex I component ND6 gene encoded by mitochondrial DNA. Complex I is one of the main sites of mitochondrial reactive oxygen species production, defects in complex I cause mitochondrial oxidative stress. Thus, experiments indicate that MEF2 regulation of mitochondrial DNA-encoded gene expression provides a direct link between aberrant Cdk5 activation and mitochondrial defects in SMA. Experiments also indicate that Cdk5 targets Ndel1 and Drp1 are involved in regulating defective mitochondrial transport and fragmentation, respectively. Cdk5 phosphorylates Ndel1 in the Lis1/Ndel1/dynein complex to activate dynein-dependent retrograde axonal transport, while hyperphosphorylation of Ndel1 by aberrant Cdk5 activation disrupts retrograde transport. In addition, Cdk5 phosphorylation of mitochondrial fission regulator Drp1 has been linked to increased mitochondrial fragmentation. These mechanisms may lead to mitochondrial retrograde transport and fragmentation defects observed in SMA motor neurons (FIG. 5). Phosphorylation of diverse targets by aberrantly activated Cdk5 may contribute to SMA pathogenesis through multiple pathways.

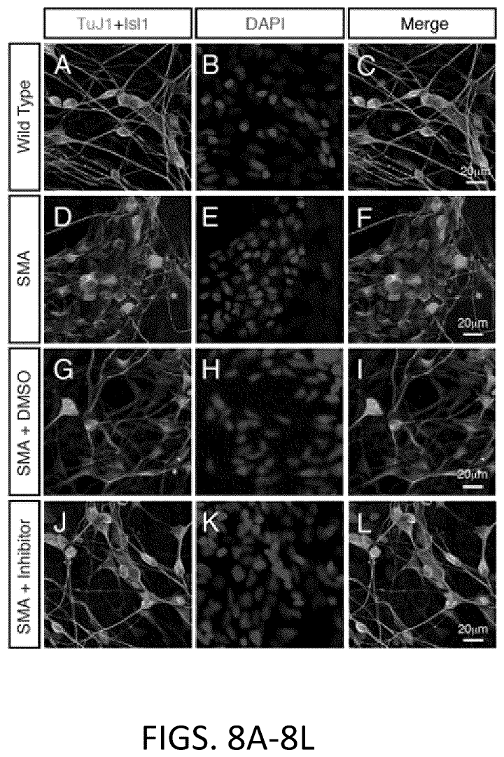

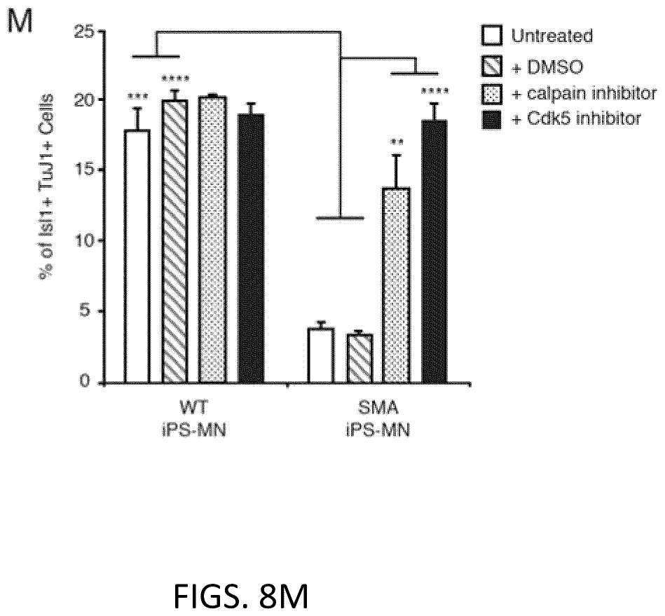

Implications for SMA Treatment. Upregulation of Cdk5 activity and the increased conversion of p35 to p25 in SMA disease are conserved between mice and humans. The finding that inhibition of Cdk5 and calpain attenuate motor neuron degeneration in human SMA iPSC-derived motor neurons (FIG. 8) indicates that inhibiting the Cdk5 signaling pathway provides benefit human patients.

Genetic knockout of p35 in SMA mice significantly rescued disease phenotypes, including mitochondrial defects, NMJ denervation and spinal motor neuron degeneration. Experiments conducted during development of embodiments herein indicate that this is due to the functional importance of Cdk5 signaling in spinal motor neurons and neural muscular junction. While the functional redundancy between p35 and Cdk5 activator p39, and the relatively high expression level of p39 in mouse spinal cord leads to the maintenance of some essential basal Cdk5 activities in the SMA;p35^{-/-} mice. Existing strategies for treating SMA are mostly based on increasing full length functional SMN protein, which has significant limitations. Inhibition of the Cdk5 signaling pathway may be used in combination with other treatments to improve motor neuron health and survival. In some embodiments, inhibitors of the Cdk5 pathway that have been developed for neurodegeneration, cancer and neuronal injury, are repurposed for treating SMA. A short peptide fragment of p35 was identified to specifically inhibit the aberrant activation of Cdk5 without affecting its normal physiological functions. Expression of this Cdk5 inhibitory peptide in mice reduces neurodegeneration in vivo without inhibiting endogenous or transfected p35/Cdk5 activity, or other cyclin-dependent kinases. In some embodiments, similar inhibitory peptides, or inhibitors that specifically disrupt the interaction between Cdk5 and its targets in SMA, are used to alleviate motor neuron disease symptoms. Mitigating aberrant Cdk5 activation is a therapeutic strategy to treat and benefit SMA patients.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

The terms “administration of” and “administering” a composition as used herein refers to providing a composition of the present disclosure to a subject in need of treatment (e.g., antiviral treatment). The compositions of the present disclosure may be administered by oral, parenteral (e.g., intramuscular, intraperitoneal, intravenous, ICV, intracisternal injection or infusion, subcutaneous injection, nebulization, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

As used herein, the terms “co-administration” and “co-administering” refer to the administration of at least two agent(s) or therapies to a subject. In some embodiments, the co-administration of two or more agents or therapies is concurrent (e.g., in a single formulation/composition or in separate formulations/compositions). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “therapeutically effective dose” refers to the amount of a pharmaceutical agent sufficient to effect beneficial or desired results. An effective amount can be administered in one or more administrations, applications or dosages and is not intended to be limited to a particular formulation or administration route.

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier and/or excipient. When a compound of the present disclosure is used contemporaneously with one or more other drugs, a pharmaceutical composition containing such other drugs in addition to the compound of the present disclosure is contemplated. Accordingly, the pharmaceutical compositions of the present disclosure include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used. Combinations of a compound of the present disclosure and other active ingredients will generally also be within the aforementioned range, but in each case, an effective dose of each active ingredient should be used. In such combinations the compound of the present disclosure and other active agents may be administered separately or in conjunction. In addition, the administration of one element may be prior to, concurrent to, or subsequent to the administration of other agent(s).

As used herein, the term “subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, macaque, etc.) and a human). In some embodiments, the subject may be a human or a non-human. In one embodiment, the subject is a human. The subject or patient may be undergoing various forms of treatment.

As used herein, the term “treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease (e.g., viral infection). A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

Embodiments of the present disclosure include compositions that comprise a Cdk5 inhibitor).

In some embodiments, the CDK5 inhibitor is an inhibit of CDK5 activity.

In some embodiments, the CDK5 inhibitor is BML-259. In some embodiments, the inhibitor is N-(5-isopropyl-thiazol-2-yl)phenylacetamide.

In some embodiments, the CDK5 inhibitor is a potent multikinase type-II inhibitor with an IC50 of 9.8 μM (e.g., CDK5-IN-4). In some embodiments, the CDK5 inhibitor is CDK5-IN-4.

In some embodiments, the CDK5 inhibitor is a brain permeable aminopyrazole analog that can inhibit Cdk5/p35 activity in the brain (e.g., 25-106). some embodiments, the CDK5 inhibitor is 25-106.

In some embodiments, the CDK5 inhibitor is a purine analog that competes with ATP-binding to CDK5 (e.g., seliciclib, roscovitine, CY-202). In some embodiments, the CDK5 inhibitor is seliciclib, roscovitine, or CY-202. In some embodiments, the CDK5 inhibitor formulation is a purine analog that competes with ATP-binding to CDK5 including tablet and capsule formulations of 100 mg, 200 mg, 300 mg, 400 mg, 500 mg, 600 mg, 700 mg, 800 mg, 900 mg, 1000 mg, in dosages of once or twice a day. In some embodiments, the CDK5 inhibitor formulation is seliciclib, roscovitine, or CY-202 including tablet and capsule formulations of 100 mg, 200 mg, 300 mg, 400 mg, 500 mg, 600 mg, 700 mg, 800 mg, 900 mg, 1000 mg, in dosages of once or twice a day.

In some embodiments, the CDK5 inhibitor bears the aminobenzimidazole moiety and displays inhibitory potential toward CDK5 activity (e.g., quinazolinone derivatives). In some embodiments, the CDK5 inhibitor is a quinazoline derivative.

In some embodiments, the CDK5 inhibitor can inhibit the pathological process of tau through the Ca2+-calpain-p25-Cdk5 pathway (e.g., quercetin). In some embodiments, the CDK5 inhibitor is quercetin.

In some embodiments, the CDK5 inhibitor is a prototype example of an inhibitor in clinical trials that targets multiple kinases including CDK5 (e.g., dinaciclib). In some embodiments, the CDK5 inhibitor is dinaciclib. In some embodiments, the CDK5 inhibitor formulation is dinaciclib including liquid formulations of 0.33 mg/m² in a dosage of a 2-hour intravenous infusion once or twice weekly for a period of 1 week, 2 weeks, 3 weeks, 4 weeks.

In some embodiments, the CDK5 inhibitor can prevent the loss of neurons and alleviate behavioral changes and/or the CDK5 inhibitor targets the hyperactivated state of CDK5, while allowing normal activation of CDK5 by p35/p39 (e.g., CDK5 inhibitory peptide (CIP)). In some embodiments, the CDK5 inhibitor is CDK5 inhibitory peptide (CIP).

In some embodiments, the CDK5 inhibitor is a derivative of a protein (e.g., a P5 peptide) that is conjugated with a cell-penetrating transactivator of transcription (TAT) sequence and a FITC tag (e.g., TFP5).

In some embodiments, the CDK5 inhibitor is a statin (e.g., a hydrophilic statin or a lipophilic statin)).

In some embodiments, the CDK5 inhibitor is metformin. In some embodiments, the CDK5 inhibitor is N, N-dimethyl amino carbon amide diamide hydrochloride. In some embodiments, the CDK5 inhibitor is Glucophage, Riomet, Fortamet, and Glumetza, Obimet, Gluformin, Dianben, Diabex, Diaformin, Metsol, Siofor, Metfogamma and Glifor. In some embodiments, the CDK5 inhibitor formulation is 100 mg/tablet of metformin, 200 mg/tablet of metformin, 300 mg/tablet of metformin, 400 mg/tablet of metformin, 500 mg/tablet of metformin, 600 mg/tablet of metformin, 700 mg/tablet of metformin, 800 mg/tablet of metformin, 900 mg/tablet of metformin, 1000 mg/tablet of metformin, 1500 mg/tablet of metformin, 2000 mg/tablet of metformin.

In some embodiments, the CDK5 inhibitor is fenofibrate (e.g., Antara, Lipofen, TriCor, Triglide, Trilipix, and generic fenofibrate). In some embodiments, the CDK5 inhibitor formulation is fenofibric acid delayed-release oral capsules. In some embodiments, the CDK5 inhibitor formulation is 20 mg of fenofibric acid delayed-release oral capsules, 25 mg of fenofibric acid delayed-release oral capsules, 30 mg of fenofibric acid delayed-release oral capsules, 35 mg of fenofibric acid delayed-release oral capsules, 40 mg of fenofibric acid delayed-release oral capsules, 45 mg of fenofibric acid delayed-release oral capsules, 50 mg of fenofibric acid delayed-release oral capsules, 120 mg of fenofibric acid delayed-release oral capsules, 125 mg of fenofibric acid delayed-release oral capsules, 130 mg of fenofibric acid delayed-release oral capsules, 135 mg of fenofibric acid delayed-release oral capsules, 140 mg of fenofibric acid delayed-release oral capsules, 145 mg of fenofibric acid delayed-release oral capsules, 150 mg of fenofibric acid delayed-release oral capsules, 155 mg of fenofibric acid delayed-release oral capsules.

In some embodiments, the CDK5 inhibitor formulation is fenofibrate oral capsules. n some embodiments, the CDK5 inhibitor formulation is 40 mg of fenofibrate oral capsules, 43 mg of fenofibrate oral capsules, 44 mg of fenofibrate oral capsules, 45 mg of fenofibrate oral capsules, 46 mg of fenofibrate oral capsules, 47 mg of fenofibrate oral capsules, 48 mg of fenofibrate oral capsules, 49 mg of fenofibrate oral capsules, 50 mg of fenofibrate oral capsules, 51 mg of fenofibrate oral capsules, 52 mg of fenofibrate oral capsules, 53 mg of fenofibrate oral capsules, 54 mg of fenofibrate oral capsules, 55 mg of fenofibrate oral capsules, 56 mg of fenofibrate oral capsules, 57 mg of fenofibrate oral capsules, 58 mg of fenofibrate oral capsules, 59 mg of fenofibrate oral capsules, 60 mg of fenofibrate oral capsules, 61 mg of fenofibrate oral capsules, 62 mg of fenofibrate oral capsules, 63 mg of fenofibrate oral capsules, 64 mg of fenofibrate oral capsules, 65 mg of fenofibrate oral capsules, 66 mg of fenofibrate oral capsules, 67 mg of fenofibrate oral capsules, 68 mg of fenofibrate oral capsules, 69 mg of fenofibrate oral capsules, 70 mg of fenofibrate oral capsules, 71 mg of fenofibrate oral capsules, 72 mg of fenofibrate oral capsules, 73 mg of fenofibrate oral capsules, 74 mg of fenofibrate oral capsules, 75 mg of fenofibrate oral capsules, 76 mg of fenofibrate oral capsules, 77 mg of fenofibrate oral capsules, 78 mg of fenofibrate oral capsules, 79 mg of fenofibrate oral capsules, 80 mg of fenofibrate oral capsules, 81 mg of fenofibrate oral capsules, 82 mg of fenofibrate oral capsules, 83 mg of fenofibrate oral capsules, 84 mg of fenofibrate oral capsules, 85 mg of fenofibrate oral capsules, 86 mg of fenofibrate oral capsules, 87 mg of fenofibrate oral capsules, 88 mg of fenofibrate oral capsules, 89 mg of fenofibrate oral capsules, 90 mg of fenofibrate oral capsules, 91 mg of fenofibrate oral capsules, 92 mg of fenofibrate oral capsules, 93 mg of fenofibrate oral capsules, 94 mg of fenofibrate oral capsules, 95 mg of fenofibrate oral capsules, 96 mg of fenofibrate oral capsules, 97 mg of fenofibrate oral capsules, 98 mg of fenofibrate oral capsules, 99 mg of fenofibrate oral capsules, 100 mg of fenofibrate oral capsules, 110 mg of fenofibrate oral capsules, 120 mg of fenofibrate oral capsules, 125 mg of fenofibrate oral capsules, 130 mg of fenofibrate oral capsules, 134 mg of fenofibrate oral capsules, 140 mg of fenofibrate oral capsules, 145 mg of fenofibrate oral capsules, 150 mg of fenofibrate oral capsules, 155 mg of fenofibrate oral capsules, 160 mg of fenofibrate oral capsules, 165 mg of fenofibrate oral capsules, 170 mg of fenofibrate oral capsules, 175 mg of fenofibrate oral capsules, 180 mg of fenofibrate oral capsules, 185 mg of fenofibrate oral capsules, 190 mg of fenofibrate oral capsules, 195 mg of fenofibrate oral capsules, 200 mg fenofibrate oral capsules.

In some embodiments, the CDK5 inhibitor formulation is fenofibrate oral tablets. In some embodiments, the CDK5 inhibitor formulation is 10 mg fenofibrate oral tablets, 20 mg fenofibrate oral tablets, 30 mg fenofibrate oral tablets, 40 mg fenofibrate oral tablets, 48 mg fenofibrate oral tablets, 50 mg fenofibrate oral tablets, 54 mg fenofibrate oral tablets, 60 mg fenofibrate oral tablets, 100 mg fenofibrate oral tablets, 110 mg fenofibrate oral tablets, 120 mg fenofibrate oral tablets, 130 mg fenofibrate oral tablets, 140 mg fenofibrate oral tablets, 145 mg fenofibrate oral tablets, 150 mg fenofibrate oral tablets, 155 mg fenofibrate oral tablets, 160 mg fenofibrate oral tablets.

In some embodiments, the CDK5 inhibitor is rosiglitazone. In some embodiments, the CDK5 inhibitor (e.g., rosiglitazone) formulation is stand-alone drug (e.g., Avandia). In some embodiments, the CDK5 inhibitor formulation is 1 mg rosiglitazone oral tablets, 2 mg rosiglitazone oral tablets, 3 mg rosiglitazone oral tablets, 4 mg rosiglitazone oral tablets, 5 mg rosiglitazone oral tablets, 6 mg rosiglitazone oral tablets, 7 mg rosiglitazone oral tablets, 8 mg rosiglitazone oral tablets, 9 mg rosiglitazone oral tablets, 10 mg rosiglitazone oral tablets. In some embodiments, the CDK5 inhibitor formulation is a combination formulation. In some embodiments, the CDK5 inhibitor formulation is a combination formulation including metformin and fenofibrate.

In some embodiments, the CDK5 inhibitor formulation is a combination formulation including metformin and fenofibrate and including metformin/fenofibrate formulations of 500 mg/1 mg, 500 mg/2 mg, 500 mg/3 mg, 500 mg/4 mg, 1000 mg/1 mg, 1000 mg/2 mg, 1000 mg/3 mg, 1000 mg/4 mg, 1500 mg/1 mg, 1500 mg/2 mg, 1500 mg/3 mg, 1500 mg/4 mg, 2000 mg/1 mg, 2000 mg/2 mg, 2000 mg/3 mg, 2000 mg/4 mg in dosages of once or twice a day. In some embodiments, the CDK5 inhibitor (e.g., rosiglitazone) formulation is in combination with metformin (e.g., Avandamet) or with glimepiride (e.g., Avandaryl).

In some embodiments, the protein is activated by neuron regulatory subunits (e.g., p35 and p39). In some embodiments, a protein/neuron regulatory subunit complex (e.g., a Cdk5/p35 complex) is activated by a calpain protease to form a different protein/neuron regulatory subunit complex (e.g., a Cdk5/p35).

In some embodiments, the CDK5 inhibitor is BML-259. In some embodiments, the inhibitor is N-(5-isopropyl-thiazol-2-yl)phenylacetamide.

In some embodiments, the CDK5 inhibitor is a purine analog that competes with ATP-binding to CDK5 (e.g., seliciclib, roscovitine, CY-202). In some embodiments, the CDK5 inhibitor is seliciclib, roscovitine, or CY-202. In some embodiments, the CDK5 inhibitor formulation is a purine analog that competes with ATP-binding to CDK5 including tablet and capsule formulations of 100 mg, 200 mg, 300 mg, 400 mg, 500 mg, 600 mg, 700 mg, 800 mg, 900 mg, 1000 mg, in dosages of once or twice a day. In some embodiments, the CDK5 inhibitor formulation is seliciclib, roscovitine, or CY-202 including tablet and capsule formulations of 100 mg, 200 mg, 300 mg, 400 mg, 500 mg, 600 mg, 700 mg, 800 mg, 900 mg, 1000 mg, in dosages of once or twice a day.

In some embodiments, the CDK5 inhibitor is dinaciclib. In some embodiments, the CDK5 inhibitor formulation is dinaciclib including liquid formulations of 0.33 mg/m² in a dosage of a 2-hour intravenous infusion once or twice weekly for a period of 1 week, 2 weeks, 3 weeks, 4 weeks.

In some embodiments, the CDK5 inhibitor targets the hyperactivated state of CDK5, while allowing normal activation of CDK5 by p35/p39 (e.g., CDK5 inhibitory peptide (CIP)). In some embodiments, the CDK5 inhibitor is CDK5 inhibitory peptide (CIP).

In some embodiments, the CDK5 inhibitor is a derivative of a protein (e.g., a P5 peptide) that is conjugated with a cell-penetrating transactivator of transcription (TAT) sequence and a FITC tag (e.g., TFP5).

In some embodiments, the CDK5 inhibitor is a statin (e.g., a hydrophilic statin or a lipophilic statin)).

In some embodiments, the CDK5 inhibitor is metformin. In some embodiments, the CDK5 inhibitor is N, N-dimethyl amino carbon amide diamide hydrochloride. In some embodiments, the CDK5 inhibitor is Glucophage, Riomet, Fortamet, and Glumetza, Obimet, Gluformin, Dianben, Diabex, Diaformin, Metsol, Siofor, Metfogamma and Glifor. In some embodiments, the CDK5 inhibitor formulation is 100 mg/tablet of metformin, 200 mg/tablet of metformin, 300 mg/tablet of metformin, 400 mg/tablet of metformin, 500 mg/tablet of metformin, 600 mg/tablet of metformin, 700 mg/tablet of metformin, 800 mg/tablet of metformin, 900 mg/tablet of metformin, 1000 mg/tablet of metformin, 1500 mg/tablet of metformin, 2000 mg/tablet of metformin.

In some embodiments, the CDK5 inhibitor is fenofibrate (e.g., Antara, Lipofen, TriCor, Triglide, Trilipix, and generic fenofibrate). In some embodiments, the CDK5 inhibitor formulation is fenofibric acid delayed-release oral capsules. In some embodiments, the CDK5 inhibitor formulation is 20 mg of fenofibric acid delayed-release oral capsules, 25 mg of fenofibric acid delayed-release oral capsules, 30 mg of fenofibric acid delayed-release oral capsules, 35 mg of fenofibric acid delayed-release oral capsules, 40 mg of fenofibric acid delayed-release oral capsules, 45 mg of fenofibric acid delayed-release oral capsules, 50 mg of fenofibric acid delayed-release oral capsules, 120 mg of fenofibric acid delayed-release oral capsules, 125 mg of fenofibric acid delayed-release oral capsules, 130 mg of fenofibric acid delayed-release oral capsules, 135 mg of fenofibric acid delayed-release oral capsules, 140 mg of fenofibric acid delayed-release oral capsules, 145 mg of fenofibric acid delayed-release oral capsules, 150 mg of fenofibric acid delayed-release oral capsules, 155 mg of fenofibric acid delayed-release oral capsules.

In some embodiments, the CDK5 inhibitor formulation is fenofibrate oral capsules. n some embodiments, the CDK5 inhibitor formulation is 40 mg of fenofibrate oral capsules, 43 mg of fenofibrate oral capsules, 44 mg of fenofibrate oral capsules, 45 mg of fenofibrate oral capsules, 46 mg of fenofibrate oral capsules, 47 mg of fenofibrate oral capsules, 48 mg of fenofibrate oral capsules, 49 mg of fenofibrate oral capsules, 50 mg of fenofibrate oral capsules, 51 mg of fenofibrate oral capsules, 52 mg of fenofibrate oral capsules, 53 mg of fenofibrate oral capsules, 54 mg of fenofibrate oral capsules, 55 mg of fenofibrate oral capsules, 56 mg of fenofibrate oral capsules, 57 mg of fenofibrate oral capsules, 58 mg of fenofibrate oral capsules, 59 mg of fenofibrate oral capsules, 60 mg of fenofibrate oral capsules, 61 mg of fenofibrate oral capsules, 62 mg of fenofibrate oral capsules, 63 mg of fenofibrate oral capsules, 64 mg of fenofibrate oral capsules, 65 mg of fenofibrate oral capsules, 66 mg of fenofibrate oral capsules, 67 mg of fenofibrate oral capsules, 68 mg of fenofibrate oral capsules, 69 mg of fenofibrate oral capsules, 70 mg of fenofibrate oral capsules, 71 mg of fenofibrate oral capsules, 72 mg of fenofibrate oral capsules, 73 mg of fenofibrate oral capsules, 74 mg of fenofibrate oral capsules, 75 mg of fenofibrate oral capsules, 76 mg of fenofibrate oral capsules, 77 mg of fenofibrate oral capsules, 78 mg of fenofibrate oral capsules, 79 mg of fenofibrate oral capsules, 80 mg of fenofibrate oral capsules, 81 mg of fenofibrate oral capsules, 82 mg of fenofibrate oral capsules, 83 mg of fenofibrate oral capsules, 84 mg of fenofibrate oral capsules, 85 mg of fenofibrate oral capsules, 86 mg of fenofibrate oral capsules, 87 mg of fenofibrate oral capsules, 88 mg of fenofibrate oral capsules, 89 mg of fenofibrate oral capsules, 90 mg of fenofibrate oral capsules, 91 mg of fenofibrate oral capsules, 92 mg of fenofibrate oral capsules, 93 mg of fenofibrate oral capsules, 94 mg of fenofibrate oral capsules, 95 mg of fenofibrate oral capsules, 96 mg of fenofibrate oral capsules, 97 mg of fenofibrate oral capsules, 98 mg of fenofibrate oral capsules, 99 mg of fenofibrate oral capsules, 100 mg of fenofibrate oral capsules, 110 mg of fenofibrate oral capsules, 120 mg of fenofibrate oral capsules, 125 mg of fenofibrate oral capsules, 130 mg of fenofibrate oral capsules, 134 mg of fenofibrate oral capsules, 140 mg of fenofibrate oral capsules, 145 mg of fenofibrate oral capsules, 150 mg of fenofibrate oral capsules, 155 mg of fenofibrate oral capsules, 160 mg of fenofibrate oral capsules, 165 mg of fenofibrate oral capsules, 170 mg of fenofibrate oral capsules, 175 mg of fenofibrate oral capsules, 180 mg of fenofibrate oral capsules, 185 mg of fenofibrate oral capsules, 190 mg of fenofibrate oral capsules, 195 mg of fenofibrate oral capsules, 200 mg fenofibrate oral capsules.

In some embodiments, the CDK5 inhibitor formulation is fenofibrate oral tablets. In some embodiments, the CDK5 inhibitor formulation is 10 mg fenofibrate oral tablets, 20 mg fenofibrate oral tablets, 30 mg fenofibrate oral tablets, 40 mg fenofibrate oral tablets, 48 mg fenofibrate oral tablets, 50 mg fenofibrate oral tablets, 54 mg fenofibrate oral tablets, 60 mg fenofibrate oral tablets, 100 mg fenofibrate oral tablets, 110 mg fenofibrate oral tablets, 120 mg fenofibrate oral tablets, 130 mg fenofibrate oral tablets, 140 mg fenofibrate oral tablets, 145 mg fenofibrate oral tablets, 150 mg fenofibrate oral tablets, 155 mg fenofibrate oral tablets, 160 mg fenofibrate oral tablets.

In some embodiments, the CDK5 inhibitor is rosiglitazone. In some embodiments, the CDK5 inhibitor (e.g., rosiglitazone) formulation is stand-alone drug (e.g., Avandia). In some embodiments, the CDK5 inhibitor formulation is 1 mg rosiglitazone oral tablets, 2 mg rosiglitazone oral tablets, 3 mg rosiglitazone oral tablets, 4 mg rosiglitazone oral tablets, 5 mg rosiglitazone oral tablets, 6 mg rosiglitazone oral tablets, 7 mg rosiglitazone oral tablets, 8 mg rosiglitazone oral tablets, 9 mg rosiglitazone oral tablets, 10 mg rosiglitazone oral tablets. In some embodiments, the CDK5 inhibitor formulation is a combination formulation. In some embodiments, the CDK5 inhibitor formulation is a combination formulation including metformin and fenofibrate.

In some embodiments, the CDK5 inhibitor formulation is a combination formulation including metformin and fenofibrate and including metformin/fenofibrate formulations of 500 mg/1 mg, 500 mg/2 mg, 500 mg/3 mg, 500 mg/4 mg, 1000 mg/1 mg, 1000 mg/2 mg, 1000 mg/3 mg, 1000 mg/4 mg, 1500 mg/1 mg, 1500 mg/2 mg, 1500 mg/3 mg, 1500 mg/4 mg, 2000 mg/1 mg, 2000 mg/2 mg, 2000 mg/3 mg, 2000 mg/4 mg in dosages of once or twice a day. In some embodiments, the CDK5 inhibitor (e.g., rosiglitazone) formulation is in combination with metformin (e.g., Avandamet) or with glimepiride (e.g., Avandaryl).

Some embodiments of the present disclosure include compositions that comprise a calpain inhibitor.

In some embodiments, the calpain inhibitor is an inhibitor of calpain activity.

In some embodiments, the calpain inhibitor is Calpeptin.

In some embodiments, the calpain inhibitor prevents the activation of AIF and reduces apoptosis in rd mice photoreceptor cells (e.g., N-acetyl-leu-leu-norleucinal (ALLN)). In some embodiments, the calpain inhibitor is N-acetyl-leu-leu-norleucinal (ALLN).

In some embodiments, the calpain inhibitor inhibits photoreceptor apoptosis in RCS rats (e.g., PD150606). In some embodiments, the calpain inhibitor is PD150606.

In some embodiments, the calpain inhibitor Penetrates the retina well when administered orally (e.g., SNJ-1945). In some embodiments, the calpain inhibitor is SNJ-1945.

In some embodiments, the calpain inhibitor Protects against focal ischemic brain damage in rats (e.g., AK275). In some embodiments, the calpain inhibitor is AK275.

In some embodiments, the calpain inhibitor Reduces the size of damaged infarct tissue in a rat focal ischemia model (e.g., MDL28170). In some embodiments, the calpain inhibitor is MDL28170.

In some embodiments, the calpain inhibitor crosses the blood-brain barrier (BBB) (e.g., Gabadur). In some embodiments, the calpain inhibitor is Gabadur.

In some embodiments, the calpain inhibitor crosses the BBB using taurine transporters (e.g., Neurodur). In some embodiments, the calpain inhibitor is Neurodur.

In some embodiments, the calpain inhibitor is An oxirane-calpain inhibitor that has shown neuroprotective effects in vivo SCI models (e.g., E-64-d). In some embodiments, the calpain inhibitor is E-64-d.

In some embodiments, the calpain inhibitor is an aldehyde-calpain inhibitor that has shown neuroprotection after (e.g., SCI Leupeptin). In some embodiments, the calpain inhibitor is SCI Leupeptin.

In some embodiments, the calpain inhibitor is potent and selective human calpain-2 inhibitor that has shown promise as a therapeutic treatment for TB (e.g., NA-184). In some embodiments, the calpain inhibitor is NA-184.

In some embodiments, provided herein is an inhibitor of the expression of a target described herein (e.g., Cdk5, calpain, p35, etc.) In some embodiments, the Cdk5, p35, and/or calpain inhibitor is an inhibitor of Cdk5, p35, and/or calpain expression. In some embodiments, the inhibitor is a small molecule, an aptamer, a siRNA, a shRNA, a miRNA, a morpholino, a ribozyme, an antisense nucleic acid molecule, a CRISPR-Cas9-based construct, a CRISPR-Cpf1-based construct, a meganuclease, a zinc finger nuclease, a transcription activator-like (TAL) effector (TALE) nuclease, etc.

In some embodiments, the inhibitor of Cdk5, p35, and/or calpain expression is a small interfering RNA (siRNA), also known as short interfering RNA or silencing RNA. In some embodiments, an siRNA is an 18 to 30 nucleotide, preferably 19 to 25 nucleotide, most preferred 21 to 23 nucleotide or even more preferably 21 nucleotide-long double-stranded RNA molecule. siRNA is involved in the RNA interference (RNAi) pathway where the siRNA interferes with the expression of a specific gene (e.g., Cdk5, p35, and/or calpain). siRNAs naturally found in nature have a well-defined structure: a short double-strand of RNA (dsRNA) with 2-nt 3′ overhangs on either end. Each strand has a 5′ phosphate group and a 3′ hydroxyl (—OH) group. This structure is the result of processing by dicer, an enzyme that converts either long dsRNAs or small hairpin RNAs into siRNAs. siRNAs can also be exogenously (artificially) introduced into cells to bring about the specific knockdown of a gene of interest (e.g., Cdk5, p35, and/or calpain). Essentially any gene for which the sequence is known can thus be targeted based on sequence complementarity with an appropriately tailored siRNA. The double-stranded RNA molecule or a metabolic processing product thereof is capable of mediating target-specific nucleic acid modifications, particularly RNA interference and/or DNA methylation. Exogenously introduced siRNAs may be devoid of overhangs at their 3′ and 5′ ends, however, in some embodiments at least one RNA strand has a 5′- and/or 3′-overhang. Preferably, one end of the double-strand has a 3′-overhang from 1 to 5 nucleotides, more preferably from 1 to 3 nucleotides and most preferably 2 nucleotides. The other end may be blunt-ended or has up to 6 nucleotides 3′-overhang. In general, any RNA molecule suitable to act as siRNA and inhibit Cdk5, p35, and/or calpain is envisioned in the present invention. In some embodiments, siRNA duplexes are provided composed of 21-nt sense and 21-nt antisense strands, paired in a manner to have a 2-nt 3′-overhang. The sequence of the 2-nt 3′ overhang makes a small contribution to the specificity of target recognition restricted to the unpaired nucleotide adjacent to the first base pair. 2′-deoxynucleotides in the 3′ overhangs are as efficient as ribonucleotides, but are often cheaper to synthesize and probably more nuclease resistant. Delivery of siRNA may be accomplished using any of the methods known in the art, for example by combining the siRNA with saline and administering the combination intravenously or intranasally or by formulating siRNA in glucose (such as for example 5% glucose) or cationic lipids and polymers can be used for siRNA delivery in vivo through systemic routes either intravenously (IV) or intraperitoneally (IP). In some embodiments, provided herein are siRNA molecules that target and inhibit the expression (e.g., knock down) of Cdk5, p35, and/or calpain.

A short hairpin RNA (shRNA) is a sequence of RNA that makes a tight hairpin turn that can be used to silence gene expression (e.g., Cdk5, p35, and/or calpain) via RNA interference. In some embodiments, shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). The RISC binds to and cleaves RNAs which match the siRNA that is bound to (e.g., comprising the sequence of Cdk5, p35, and/or calpain). In some embodiments, si/shRNAs to be used in the present invention are chemically synthesized using appropriately protected ribonucleoside phosphoramidites and a conventional DNA/RNA synthesizer. In some embodiments, provided herein are shRNA molecules that target and inhibit the expression (e.g., knock down) of Cdk5, p35, and/or calpain.

Further molecules effecting RNAi (and useful herein for the inhibition of expression of Cdk5, p35, and/or calpain) include, for example, microRNAs (miRNA). Said RNA species are single-stranded RNA molecules. Endogenously present miRNA molecules regulate gene expression by binding to a complementary mRNA transcript and triggering of the degradation of said mRNA transcript through a process similar to RNA interference. Accordingly, exogenous miRNA may be employed as an inhibitor of Cdk5, p35, and/or calpain after introduction into target cells. In some embodiments, provided herein are miRNA molecules that target and inhibit the expression (e.g., knock down) of the Cdk5, p35, and/or calpain.

Morpholinos (or morpholino oligonucleotides) are synthetic nucleic acid molecules having a length of about 20 to 30 nucleotides and, typically about 25 nucleotides. Morpholinos bind to complementary sequences of target transcripts (e.g., Cdk5, p35, and/or calpain) by standard nucleic acid base-pairing. They have standard nucleic acid bases which are bound to morpholine rings instead of deoxyribose rings and linked through phosphorodiamidate groups instead of phosphates. Due to replacement of anionic phosphates into the uncharged phosphorodiamidate groups, ionization in the usual physiological pH range is prevented, so that morpholinos in organisms or cells are uncharged molecules. The entire backbone of a morpholino is made from these modified subunits. Unlike inhibitory small RNA molecules, morpholinos do not degrade their target RNA molecules. Rather, they sterically block binding to a target sequence within a RNA and prevent access by molecules that might otherwise interact with the RNA. In some embodiments, provided herein are morpholino oligonucleotides that target and inhibit the expression (e.g., knock down) of Cdk5, p35, and/or calpain.

A ribozyme (ribonucleic acid enzyme, also called RNA enzyme or catalytic RNA) is an RNA molecule that catalyzes a chemical reaction. Many natural ribozymes catalyze either their own cleavage or the cleavage of other RNAs, but they have also been found to catalyze the aminotransferase activity of the ribosome. Non-limiting examples of well-characterized small self-cleaving RNAs are the hammerhead, hairpin, hepatitis delta virus, and in vitro-selected lead-dependent ribozymes, whereas the group I intron is an example for larger ribozymes. The principle of catalytic self-cleavage is well established. Since it was shown that hammerhead structures can be integrated into heterologous RNA sequences and that ribozyme activity can thereby be transferred to these molecules, catalytic antisense sequences can be engineered for almost any target sequence can be created, provided the target sequence contains a potential matching cleavage site. The basic principle of constructing hammerhead ribozymes is as follows: A region of interest of the RNA (e.g., a portion of Cdk5, p35, and/or calpain), which contains the GUC (or CUC) triplet, is selected. Two oligonucleotide strands, each usually with 6 to 8 nucleotides, are taken and the catalytic hammerhead sequence is inserted between them. In some embodiments, provided herein are ribozyme inhibitors of the Cdk5, p35, and/or calpain.

In some embodiments, Cdk5, p35, and/or calpain is inhibited by modifying the Cdk5, p35, and/or calpain sequence in target cells. In some embodiments, the alteration of Cdk5, p35, and/or calpain is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus. The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes. In some aspects, a Cas nuclease and gRNA (including a fusion of crRNA specific for the target sequence (e.g., a sequence within Cdk5, p35, and/or calpain) and fixed tracrRNA) are introduced into the cell. In general, target sites at the 5′ end of the gRNA target the Cas nuclease to the target site using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence (e.g., sequence within the Cdk5, p35, and/or calpain gene). In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex. The CRISPR system can induce double stranded breaks (DSBs) at the SRC-3 target site, followed by disruptions or alterations as discussed herein. In other embodiments, Cas9 variants, deemed “nickases,” are used to nick a single strand at the target site (e.g., within the Cdk5, p35, and/or calpain gene). Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression (e.g., to inhibit expression of Cdk5, p35, and/or calpain). In some embodiments, the CRISPR system is used to alter Cdk5, p35, and/or calpain, inhibit expression of Cdk5, p35, and/or calpain, and/or to inactivate the expression product of the Cdk5, p35, and/or calpain.

The term “antisense nucleic acid molecule” or “antisense oligonucleotide” as used herein, refers to a nucleic acid which is complementary to a target nucleic acid. An antisense molecule in accordance with the invention is capable of interacting with the target nucleic acid, more specifically it is capable of hybridizing with the target nucleic acid. Due to the formation of the hybrid, transcription of the target gene(s) and/or translation of the target mRNA is reduced or blocked. Standard methods relating to antisense technology have been described (see, e.g., Melani et al., Cancer Res. (1991) 51:2897-2901). In some embodiments, provided herein are antisense oligonucleotides capable of inhibiting expression of Cdk5, p35, and/or calpain when administered to cell or subject. In some embodiments, the antisense oligonucleotides are antisense DNA- and/or RNA-oligonucleotides. In some embodiments, provided herein are modified antisense oligonucleotides, such as, antisense 2′-O-methyl oligo-ribonucleotides, antisense oligonucleotides containing phosphorothiaote linkages, antisense oligonucleotides containing Locked Nucleic Acid LNA(R) bases, morpholino antisense oligonucleotides, PPAR-gamma agonists, antagomirs. In some embodiments, ASOs comprise Locked Nucleic Acid (LNA) or 2′-methoxyethyl (MOE) modifications (internucleotide linkages are phosphorothioates interspersed with phosphodiesters, and all cytosine residues are 5′-methylcytosines).

In some embodiments, the calpain inhibitor can mitigate Parkinson disease (PD) neuropathology. In some embodiments, the calpain inhibitor can attenuate the progression of PD neuropathology.

In some embodiments, the CDK5 inhibitor formulation and/or the calpain inhibitor formulation is a tablet. In some embodiments, the CDK5 inhibitor formulation and/or the calpain inhibitor formulation is an immediate release tablet. In some embodiments, the CDK5 inhibitor formulation and/or the calpain inhibitor formulation is an orodispersibie tablet. In some embodiments, the CDK5 inhibitor formulation and/or the calpain inhibitor formulation is released immediately upon contact with the wet mucosa surface of the oral cavity and requires no water or chewing before ingestion. In some embodiments, the CDK5 inhibitor formulation and/or the calpain inhibitor formulation is a tablet manufactured using compression.

In some embodiments, the CDK5 inhibitor formulation and/or the calpain inhibitor formulation is a tablet including a mixture of any one or more of: a disintegrant (e.g., an additive that promotes disintegration (i.e., the breakage of a tablet into small fragments when in contact with a liquid medium) (e.g., starch USP, starch 1500, microcrystalline cellulose (Avicel), alginic acid, guar gum, methylcellulose, sodium carboxymethylcellulose, sodium starch glycolate (SSG), croscarmellose sodium (CCS), and pregelatinized starch), a super disintegrant (e.g., a disintegrant used at a low level (e.g., 1%-10%) by weight basis as compared to a conventional disintegrant in the solid dosage form)(e.g., sodium starch glycolate (e.g., Explotab, Primogel), cross-linked polyvinyl-pyrrolidone (e.g., crospovidone, PolyplasdoneXL, PolyplasdoneXL10), croscarmellose sodium (e.g., Ac-Di-Sol), and magnesium aluminum silicate (e.g., Veegum HV), and a sugar-based excipient (e.g., mannitol).

In some embodiments, the CDK5 inhibitor formulation and/or the calpain inhibitor formulation is a tablet including a disintegrant or a super disintegrant, wherein the disintegrant or a super disintegrant is added prior to compression (e.g., internal addition (e.g., the disintegrant is mixed with other powders before granulation), external addition (e.g., the disintegrant is added to the granules before compression (e.g., mixing before compression)), and combination method (e.g., both internal and external additions of disintegrants are used)).

In some embodiments, the CDK5 inhibitor formulation and/or the calpain inhibitor formulation is a liquid.

Embodiments of the present disclosure also include methods of treating a subject who suffers from a pediatric neuromuscular disease (e.g., SMA) comprising administering any of the compositions described herein to a subject in need thereof. In some embodiments, the pediatric neuromuscular disease is SMA.

Embodiments of the present disclosure also include methods of treating a subject who suffers from a neurodegenerative disorder (e.g., neurodegenerative disorders with dysregulation of Cdk5 signaling and mitochondrial defects) comprising administering any of the compositions described herein to a subject in need thereof. In some embodiments, the neurodegenerative disorder is Alzheimer's Disease, Parkinson disease (PD) neuropathology, Amyotrophic lateral sclerosis (ALS), and/or Huntington's disease. In some embodiments, the neurodegenerative disorder is a chronic disease that causes the central nervous system to progressively lose myelin sheath (e.g., Multiple sclerosis (MS)). In some embodiments, the neurodegenerative disorder is a syndrome or condition that can be caused by a number of diseases (e.g., Dementia). In some embodiments, the neurodegenerative disorder is a neurodegenerative condition that can only be diagnosed after death through a microscopic tissue analysis of the brain (e.g., Pick's disease, chronic traumatic encephalopathy).

In some embodiments, the composition is administered orally, parenterally, intramuscularly, intraperitoneally, intravenously, intracerebroventricularly, intracisternally, intratracheally, intranasally, subcutaneously, via injection or infusion, via inhalation, spray, nasal, vaginal, rectal, sublingual, or topical administration. In some embodiments, the composition is administered via nebulization to lung tissue.

Embodiments of the present disclosure also include a method of treating a subject who suffers from a pediatric neuromuscular disease (e.g., SMA) comprising targeting the Cdk5 signaling pathway. In some embodiments, the method includes inhibiting Cdk5 activation. In some embodiments, the method includes reducing Cdk5 activation. In some embodiments, the method includes improving mitochondrial function (e.g., rescuing mitochondrial transport and fragmentation; reducing mitochondrial oxidative stress; aberrant Cdk5 activation) by inhibiting the expression or activity of p35.

Embodiments of the present disclosure also include methods of treating a subject who suffers from other neurodegenerative disorders (e.g., neurodegenerative disorders with dysregulation of Cdk5 signaling and mitochondrial defects) comprising targeting the Cdk5 signaling pathway. In some embodiments, the method includes inhibiting Cdk5 activation. In some embodiments, the method includes reducing Cdk5 activation. In some embodiments, the method includes improving mitochondrial function (e.g., rescuing mitochondrial transport and fragmentation; reducing mitochondrial oxidative stress; aberrant Cdk5 activation) by inhibiting the expression or activity of p35.

### Experimental

### Material and Methods

Mice. The Δ7 SMA mice (Smn^{-/-};SMN2′^{tg/tg};SMNΔ7^{tg/tg}, or Jackson Lab #005025), and the Hung-Li SMA mice (Sm^{+/-};SMN2Hung^{tg/tg}, or Jackson Lab #005058) were obtained from Jackson Laboratory. Pups were genotyped by PCR with genomic DNA extracted from tail samples. The P35 knockout mice were generated and provided by Dr. Li-Huei Tsai's laboratory and genotyped as reported. Both male and female mice were used. ARRIVE guidelines were followed for the animal experiments. Investigators were blinded to the treatment groups.

Human SMA iPSC and Mouse Motor Neuron Culture. Human iPSCs were generated and differentiated as described in the Examples. Briefly, primary fibroblasts were isolated from de-identified type I SMA patients and their unaffected relatives, infected with lentiviral constructs expressing OCT4, SOX2, NANOG, and LIN28 or episomally expressed reprogramming plasmids to generate iPSCs. Retinoic acid, sonic hedgehog, cAMP, ascorbic acid, brain-derived neurotrophic factor (BDNF) and glial cell line-derived neurotrophic factor (GDNF) were used to induce iPSC spheres to differentiate into motor neurons. Primary neurons from mouse spinal cords were cultured in Neurobasal (Life Technologies) supplemented with B27 (Life Technologies) as described. Briefly, spinal cords from E12.5 mouse embryos were dissected out and dissociated with 0.25% trypsin. After enriching motor neurons with Optiprep density gradient centrifugation and BSA cushion, cells were seeded on glass cover slips coated with 20 μg/ml Poly-L-Lysine (PLL) (Sigma) and 8 μg/ml Laminin (Sigma), and grown in the presence of 50 μg/ml BDNF, 50 μg/ml CNTF and 25 μg/ml GDNF (PeproTech). Cell death was detected by the in situ cell death detection TMR red TUNEL (Terminal Transferase dUTP Nick End Labeling) kit. Cdk5 inhibitor BML259 (300 nM) (Cayman Chemical) and calpain inhibitor Calpeptin (10 μM) (Tocris Bioscience) were applied from week 3 to 6 into iPSC motor neuron medium to inhibit Cdk5 signaling.

In vitro Cdk5 Kinase Assay. Endogenous Cdk5 kinase in spinal cord lysates was immunoprecipitated with anti-Cdk5 antibodies (C17 monoclonal or Cell Signaling #2506). The Cdk5 kinase immunopricipitation complexes were washed and suspended in kinase buffer containing 50 mM Hepes (pH7.4), 5 mM MnCl₂, 5 mM MgCl₂, 2 mM DTT, and incubated with 2 μg purified Histone H1 (Sigma) in the presence of 1 μM ATP and 10 μM γ-³²P-ATP at 30° C. for 15 min. Samples were then denatured in SDS sample buffer, separated on SDS-PAGE, and subjected to autoradiography, scintillation counting, and phosphor imager quantification. Relative endogenous Cdk5 activity was calculated by dividing the Cdk5 kinase activity of each SMA spinal cord sample with the kinase activity of its paired wild type sample.

Confocal Live Imaging and Data Analysis of Mitochondria Transport and Length. Time-lapse live imaging by confocal microscope was used to measure axonal mitochondrial transport and length. After culturing for 5-7 days, primary mouse spinal motor neurons were transfected with mitochondria targeting sequence-tagged DsRed (mito-DsRed). 48 hours after transfection, images were acquired using a Zeiss LSM 700 confocal microscope equipped with a 63×/NA 1.15 water LD C-Apochromat objective lens and a temperature (37° C.) and CO₂ (5%) controlled stage. Images were captured every 2 seconds for a period of 2 minutes using Zen software. The 561 nm laser intensity was set at 0.2 mW to minimize damage, and pinholes were opened maximally to allow the entire thickness of the axon to be imaged. Axon fragments of 50-100 μm in length located at least 50 μm away from the cell body were selected for analysis. A custom-made Image J plug-ins was used to generate kymographs and analyze mitochondria motility and length.

Electrophysiological Recording. Mice at P7-8 were deeply anesthetized with isofluorane, decapitated and eviscerated. The lower thoracic to upper lumbar spinal cord was removed and embedded in 2.5% w/v agar. Using the Leica 1000 vibratome 350 μm transverse slices were made as described. During spinal cord isolation and slicing, the spinal cord was immersed in 1-4° C. high osmolarity dissecting solution containing (in mM) sucrose 234.0, KCl 2.5, CaCl₂·2H₂O 0.1, MgSO₄·7H₂O 4.0, HEPES 15.0, glucose 11.0, Na₂PO₄ 1.0. After cutting, the slices were incubated for >1 hour at 30° C. in incubating solution containing (in mM) NaCl 126.0, KCl 2.5, CaCl₂·2H₂O 2.0, MgCl₂·6H₂O 2.0, NaHCO₃ 26.0, glucose 10.0. Whole cell patch clamp was performed at room temperature on motor neurons using 2-5 MΩ glass electrodes positioned using a Sutter Instrument MP-285 micromanipulator. Slices were perfused with a modified Ringer's solution containing (in mM): 111 NaCl, 3.09 KCl, 25.0 NaHCO₃, 1.10 KH₂PO₄, 1.26 MgSO₄, 2.52 CaCl₂, and 11.1 glucose. All solutions were continuously oxygenated with 95% O₂ and 5% CO₂. Recordings were performed in current and voltage clamp modes as described using the Multiclamp 700B amplifier (Molecular Devices, Burlingame, CA). Patch electrodes contained (in mM) 138 K-gluconate, 10 HEPES, 5 ATP-Mg, and 0.3 GTP-Li (all from Sigma, St. Louis, MO). In voltage clamp mode, neurons were subjected to slow, depolarizing voltage ramps to measure the persistent inward current (PIC) bringing the cell from −90 mV holding potential to 0 mV in 8 s, and then back to −90 mV in the following 8 s. In current clamp, neurons were subjected to depolarizing current ramps to measure ION (the current level at firing onset), IOFF (the current level at cessation of firing) and the frequency-current relationship. The medial motor neuron pools could be easily visualized in the slice preparation using DIC optics, and electrodes were positioned above this area. Individual neurons were targeted based on large soma diameter (>20 μM long axis) and only neurons with an input resistance <100MΩ, a resting potential <−50 mV, and a series resistance of <20 MΩ were included in this study. Neurons were eliminated from analysis if series resistance or resting potential varied more than 10 MΩ or 10 mV, respectively, throughout the recording period. Data was collected on Winfluor software (University of Strathclyde, Glasgow, Scotland) and analyzed using Spike2 software (Cambridge Electronic Design, Cambridge, England). Graphs and statistics were performed using Microsoft Excel and GraphPad Prism.

Lentiviral shRNA and Real-Time PCR. Lentivirus expressing shRNA for Cdk5 and scrambled control were purchase from Santa Cruz Biotechnologies. For real-time quantitative PCR assay, total RNA was extracted from homogenized mouse spinal cord with the RNeasy kit (Qiagen) and treated with RNase-free DNase set (Qiagen). For cDNA synthesis, 1 μg of total RNA was used for reverse transcription using the oligo(dT)-primer and SuperScript III kit (Invitrogen). Real-time PCR was performed using SYBR Green PCR Master Mix (Applied Biosystems) and measured with Applied Biosystems 7500 Real-Time PCR System. Primers used for the RT-qPCR are: bdnf: Forward (F) GATGCCGCAAACATGTCTATGA, Reverse (R) TAATACTGTCACACACGCTCAGCTC; gapdh: (F) CATGGCCTTCCGTGTTCCT, (R) TGATGTCATCATACTTGGCAGGTT; actin: (F) GCGAGCACAGCTTCTTTGC, (R) TCGTCATCCATGGCGAACT; tubulin: (F) CGACAATGAAGCCCTCTACGAC, (R) ATGGTGGCAGACACAAGGTGGTTG.

Immunohistochemistry. SMA and wild type (WT) mice were perfused with PBS and 4% PFA. Spinal cords were isolated and fixed overnight in freshly made 4% PFA. All samples were washed extensively with PBS and treated sequentially with 15% and 30% sucrose before embedding into OCT. Cryosections of 18 μm thick were then prepared with a Leica CM1950 cryostat. For immunostaining, tissue sections were first treated with 10 mM citric acid antigen retrieval solution (DAKO) at 98° C. for 20 minutes, then permeabilized in 0.25% Triton X-100 and blocked with 5% donkey serum and 5% goat serum in PBST buffer (PBS with 0.05% Tween-20). Samples were then incubated with primary antibodies overnight at 4° C., washed with PBST, incubated with secondary antibodies, washed with PBST, mounted in Aquamount (Fisher Scientific), and imaged with Zeiss LSM510 confocal microscope. Primary antibodies used in this study are as follows: p35/p25 (1:500, Cell Signaling #2680 rabbit monoclonal), Isl1 (1:750, Developmental Studies Hybridoma Bank 40.2D6 mouse monoclonal), SV2 (1:10, Developmental Studies Hybridoma Bank mouse monoclonal), ChAT (1:100, Chemicon AB144P goat polyclonal), vGluT1 (1:500, Synaptic System 135303, rabbit polyclonal), neurofilament (1:100, Developmental Studies Hybridoma Bank 2H3 mouse monoclonal), α-bungarotoxin (1:1000, Invitrogen, Alexa 594-conjugated), TuJ1 (1:1000, Covance MRB-435Prabit polyclonal). Secondary antibodies are from Jackson ImmunoResearch and used at 1:500 dilution.

NMJs of mouse flexor digitorum brevis (FDB) muscles were stained. Briefly, FDB muscles were isolated and teased into layers of 5-10 fibers thick to facilitate penetration of antibodies. Presynaptic nerve terminals were labeled with anti-neurofilament and anti-SV2antibodies. Acetylcholine receptors (AChRs) were labeled by α-bungarotoxin. Z-stack images of fluorescently labeled NMJs were captured at sequential focal planes 1p m apart using a Zeiss LSM 510 META confocal microscope. All confocal images were taken using the same imaging parameters, including laser intensities, amplification gains and offsets. To study excitatory synapse formation on spinal motor neurons, lumbar spinal cord segments (L1-2) were dissected and processed for 80 μm thick vibratome (Leica) sections. Motor neurons were labeled with anti-choline acetyltransferase (ChAT) antibody and excitatory presynaptic terminals with anti-vesicular glutamate transporter 1 (vGluT1). Glutamatergic synapses were identified as boutons opposed to membrane of motor neuron soma and proximal dendrites. The number of synapses from Z-stack images of the whole motor neuron soma was counted. Statistical significance was determined using one-way ANOVA analysis with Tukey HSD post hoc analysis.

Western Blotting and Immunoprecipitation. Spinal cord samples or motor neurons were lysed in the buffer containing 200 mM NaCl, 0.4% Triton X-100, 0.7% CHAPS, 50 mM Tris pH8.0, 5 mM EDTA, 5 mMDTT plus proteinase inhibitors to be used for Western blotting or immunoprecipitation. Samples were homogenized by a polytron handheld homogenizer (Kinematica) and quantified using the BCA protein Assay kit (Pierce). All proteins were separated with 10% SDS-PAGE, then transferred to PVDF membrane and incubated with indicated antibodies. Primary antibodies were diluted in TBS containing 0.05% Tween-20 and 5% bovine serum albumin as follows: Cdk5 (1:5, Dr. Li-Huei Tsai C-17 mouse monoclonal), p35/p25 (1:500, Cell Signaling #2680 rabbit monoclonal), GSK3P (1:500, Cell Signaling #12456 rabbit monoclonal), GAPDH (1:1000, Santa Cruz Biotech SC-25778 rabbit polyclonal), Histone H1 (1:500, Santa Cruz Biotech SC-8030 mouse monoclonal). Horseradish peroxidase (HRP)-conjugated secondary antibodies (1:10,000, Jackson ImmunoResearch) and Femto LUCENT plus HRP reagent kit (G Biosciences) were used for exposure and quantification.

### EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

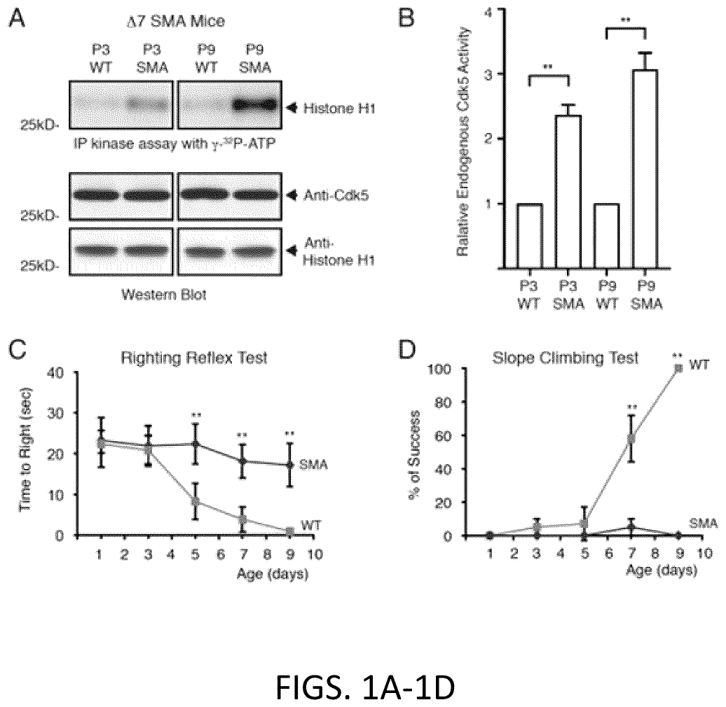

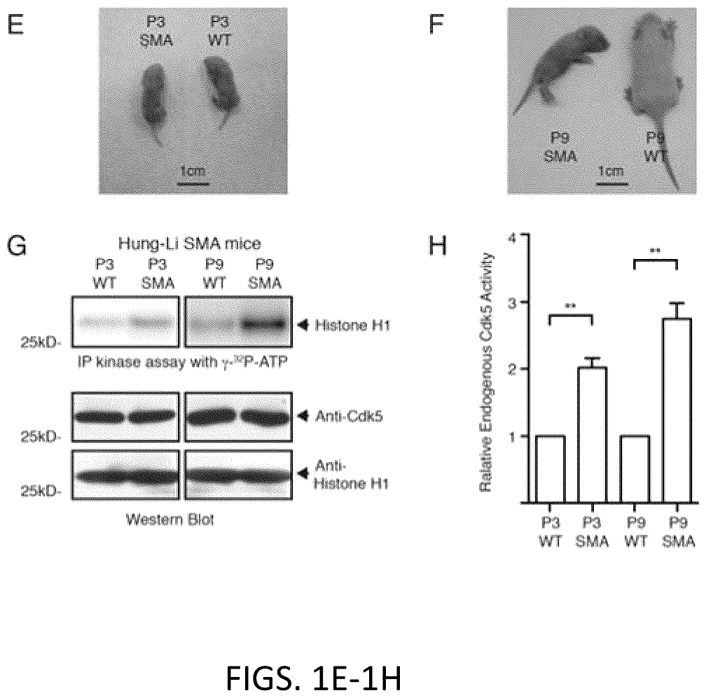

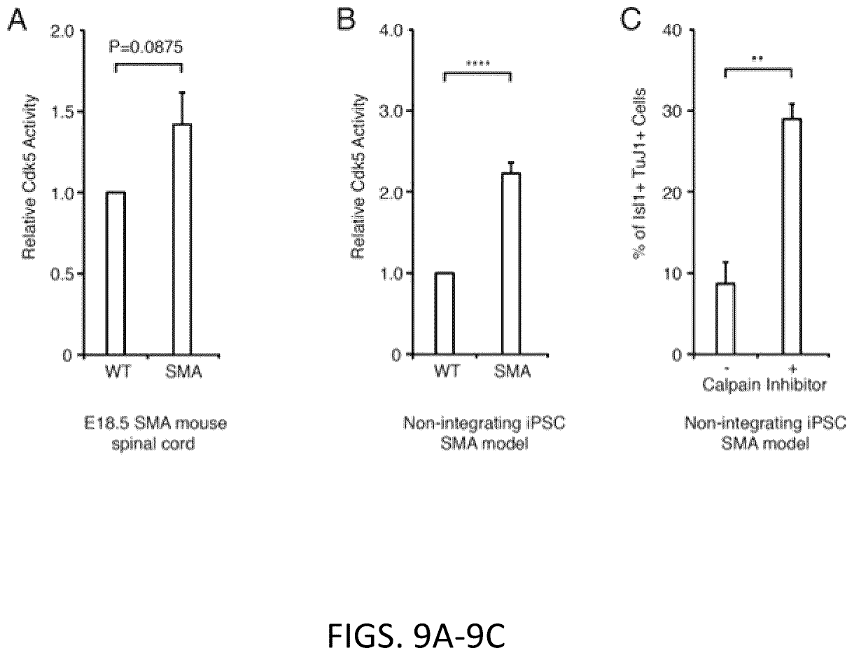

Aberrant Activation of Cdk5 Kinase in SMA Mice Before the Onset of Disease Symptoms. Increased Cdk5 kinase activity has been implicated in a number of neurodegenerative disorders. Because SMA is characterized by spinal motor neuron degeneration, whether the activity of Cdk5 kinase is dysregulated in SMA disease conditions was examined. Two widely used SMA mouse models were used, the Δ7 SMA mice (Smn^{-/-};SMN2^{tg/tg};SMNΔ7^{tg/tg}) and the Hung-Li SMA mice (Smn^{-/-};SMN2Hung^{tg/-}). These mouse models express different forms of human SMN2 transgene on the mouse Smn knockout background. Disease mice have an average life span of approximately 13 days and exhibit symptoms and neuropathology similar to patients afflicted with severe forms of SMA. Using the Δ7 SMA mice, endogenous Cdk5 kinase was immunoprecipitated from spinal cord lysates and its activity of phosphorylating a known Cdk5 substrate, Histone H1, was assayed with ³²P-γ-ATP. It was found that endogenous Cdk5 kinase activity was upregulated in SMA mice compared to control wild type littermates at postnatal day 9 (P9) (FIGS. 1A, 1B), while the amount of Cdk5 protein was at the same level (FIG. 1A). At P9, the Δ7 SMA mice showed severe motor behavioral defects (FIGS. 1C, 1D) and were smaller than the wild type littermates (FIG. 1F). Therefore, P9 was considered a post-symptomatic stage. To test whether the upregulation of Cdk5 kinase activity is a secondary effect to motor neuron degeneration in SMA mice, Cdk5 activity at postnatal day 3 (P3) was examined. P3 is a pre-symptomatic stage when Δ7 SMA mice are not significantly different from their wild type littermates (FIGS. 1C-1E). A substantial increase in endogenous Cdk5 kinase activity in pre-symptomatic P3 mice (FIGS. 1A, 1B) was observed, indicating that the increase of Cdk5 kinase activity is not a consequence of disease phenotypes in SMA, but instead contributes to the initiation of SMA pathogenesis. Similarly, endogenous Cdk5 activity was also found to be significantly increased in a second SMA mouse model, the Hung-Li SMA mice (FIGS. 1G, 1H). These observations indicate that increased Cdk5 activity is not limited to a specific SMA mouse model but is associated with the general SMA pathology. Furthermore, Cdk5 activity was also examined at earlier time points (E18.5) in SMA mouse models and increased Cdk5 kinase activity was found, although not statistically significant (FIG. 9A). Taken together, these results indicate that the upregulation of Cdk5 kinase activity in the spinal cord is a conserved phenomenon that contributes to the initiation of SMA pathogenesis.

### Example 2

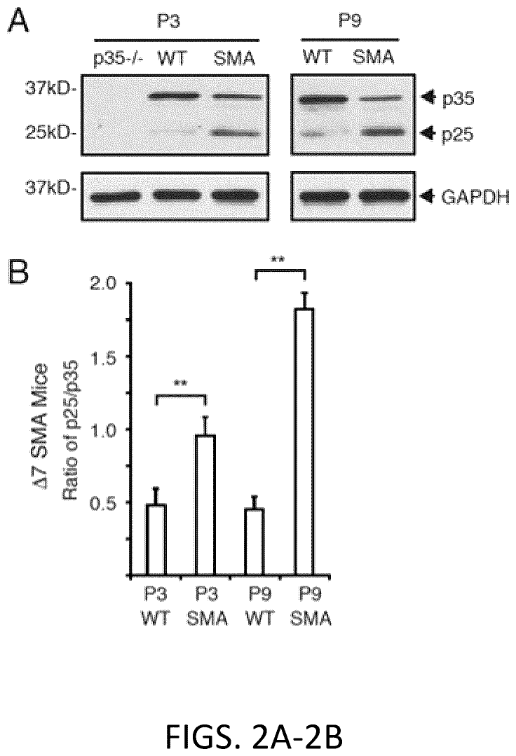

Increased Conversion of p35 to p25 in SMA Mouse Models. The kinase activity of Cdk5 is dictated by its activating subunit p35 or p25. P35 can be cleaved by Ca²⁺-dependent protease calpain to generate p25, which is a more potent Cdk5 activator. The observation that Cdk5 protein levels were not changed in SMA mice (FIGS. 1A, 1G) prompted a test of whether the upregulated Cdk5 kinase activity was due to increased conversion of p35 into p25. The levels of p35 and p25 in spinal cords from both the Δ7 SMA mice and the Hung-Li SMA mice were examined by Western blot analysis. Specificity of the anti-p35/p25 antibody was confirmed on neuronal lysates from p35 knockout mice (FIG. 2A). Increased p25 protein levels in spinal cords of Δ7 SMA mice (FIGS. 2A, 2B) and Hung-Li SMA mice (FIG. 2C) was found, compared to those of littermate control mice at P9. To test whether the upregulation of p25 occurred before SMA phenotypes, the level of p25 at the pre-symptomatic stage P3 was also examined. Increased p25 in P3 SMA spinal cords of both Δ7 SMA mice (FIGS. 2A, 2B) and Hung-Li SMA mice (FIG. 9D) was found. These data indicate that calpain-mediated conversion of p35 to p25 is also upregulated in SMA mouse models before the onset of disease symptoms, leading to aberrant Cdk5 kinase activation.

### Example 3

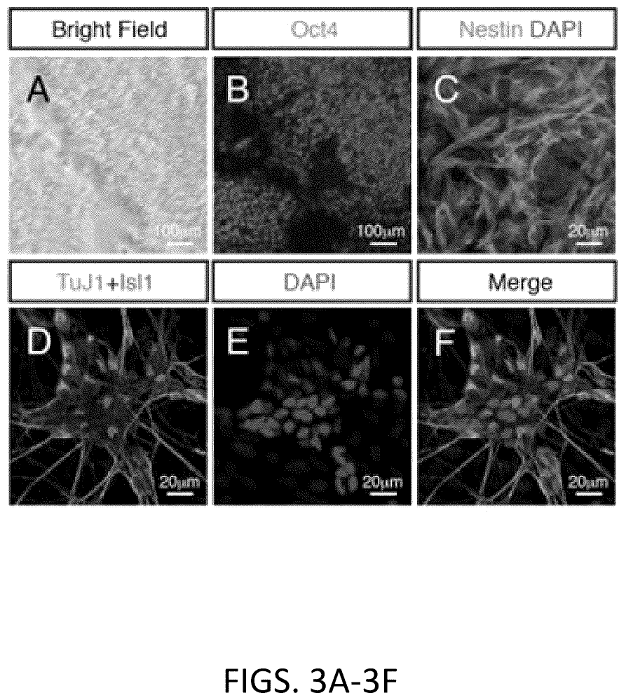

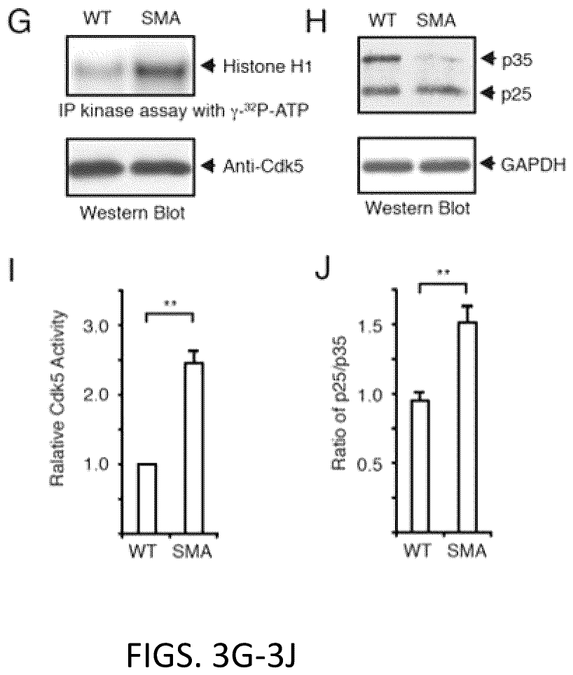

Upregulations of Cdk5 Activity and p25 Level Are Conserved in Human iPSC Models of SMA. Experiments were conducted during development of embodiments herein to determine whether increased conversion of p35 to p25 and the upregulated Cdk5 activity in SMA mice exist in human SMA disease. iPSCs derived from human SMA patients were used. Patient-derived iPSCs offer unique advantages in studying disease mechanisms and performing drug screenings because of their identical genetic architecture to human patients. SMA iPSCs are generated from skin fibroblasts of SMA patients and their unaffected relatives. Motor neurons that express both neuronal marker TuJ1 and motor neuron marker Isl1 are derived from Oct4-expressing pluripotent SMA iPSCs (FIGS. 3A-3F). These SMA iPSC-derived motor neurons show increased caspase activation, motor neuron degeneration and other disease phenotypes (61-63), serving as a human iPSC model of SMA. Consistent with results from SMA mice, immunoprecipitation kinase assay showed substantially increased endogenous Cdk5 kinase activity in motor neurons derived from SMA iPSCs generated by lentivirus-based reprograming (FIGS. 3G, 3I) or by footprint-free non-integrating episomal approach (FIG. 9B). In addition, quantification of Western blot analysis revealed that p25 levels also increased in SMA iPSC-derived motor neurons (FIGS. 3H, 3J), indicating that upregulations of Cdk5 activity and p25 level in SMA pathogenesis are a mechanism conserved in human patients.

### Example 4

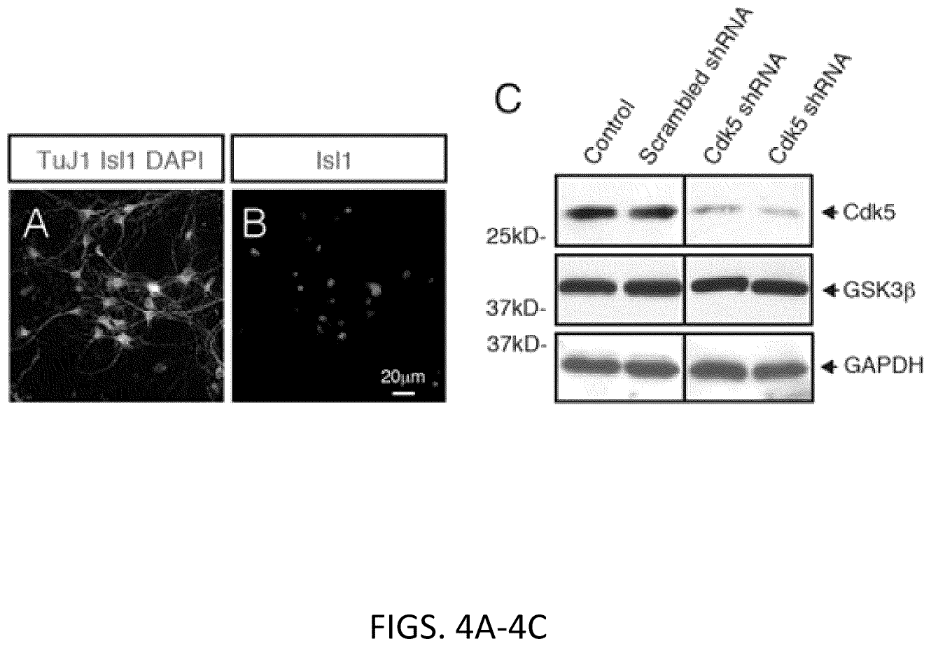

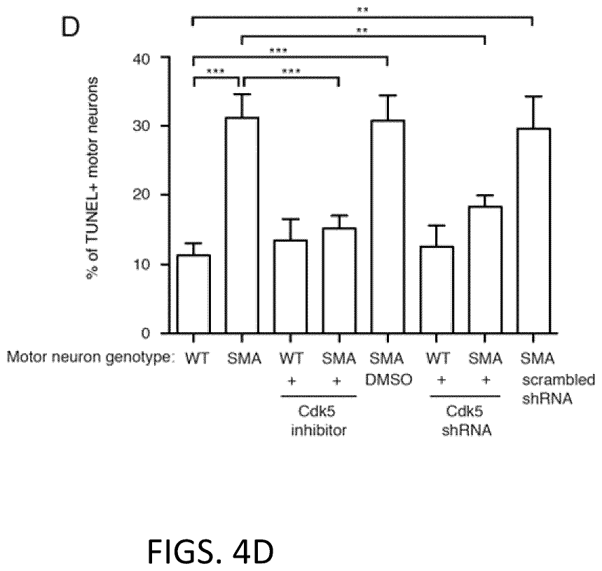

Mitigating Aberrant Cdk5 Activation Reduces the Degeneration of SMA Mouse Motor Neurons. Experiments were conducted during development of embodiments herein to determine whether reducing aberrant Cdk5 activation in SMA conditions serve to ameliorate motor neuron degeneration; the degeneration of motor neurons cultured from Δ7 SMA mice and control mice by TUNEL staining was monitored. These mouse motor neurons were treated with lentivirus carrying short hairpin RNA (shRNA) to knock down Cdk5 expression, or pharmacological inhibitors to reduce Cdk5 kinase activity (FIG. 4). Isl1 and TuJ1 positive motor neurons from Δ7 SMA mice showed dramatically increased degeneration compared to those from non-disease control mice (FIG. 4D), while applying 300 nM Cdk5 inhibitor BML259 or shRNA-mediated Cdk5 knockdown significantly reduced degeneration (FIG. 4D). These data support a critical role for aberrant Cdk5 activation in causing motor neuron degeneration in SMA.

TABLE 2
Dosage effect of BML259 on primary mouse motor neurons.

| BML 259 | 50 nM | 150 nM | 300 nM | 450 nM | 600 nM |
|---|---|---|---|---|---|
| Toxic Effect on | no | no | no | weak | strong |
| WT motor neuron | | | | | |
| Toxic Effect on | no | no | no | strong | very strong |
| SMA motor neuron | | | | | |

Different concentrations of Cdk5 inhibitor BML259 were applied on primary motor neurons from wild type and SMA mice. After two days, treated and untreated cells were compared to identify potential toxic effects of causing additional cell death by checking cell morphology and viability using a bright field microscope.

### Example 5

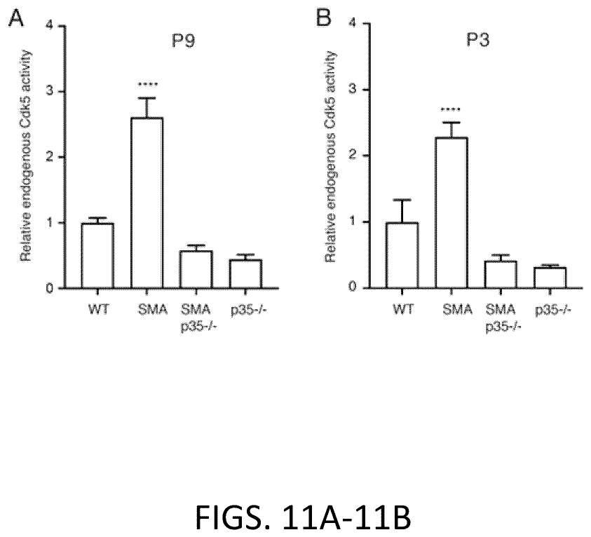

Genetic Knockout of p35 Rescues Mitochondrial Defects in SMA Mouse Motor Neurons. To further establish the importance of Cdk5 signaling in SMA pathogenesis, experiments were conducted during development of embodiments herein to determine whether genetic knockout of Cdk5 or its activating subunit p35 can rescue functional defects in SMA was tested. This required the generation of compound mutant mice that carry the Cdk5 or p35 knockout alleles on the background of a SMA mouse model. Cdk5 null mice are perinatal lethal, preventing the analysis of SMA phenotypes in postnatal mice. Therefore, p35 null mice, which can survive into adulthood (65), were crossed with the Hung-Li SMA mouse model (Smn^{-/-}; SMN2Hung^{tg/-}) to generate a compound Smn^{-/-}; SMN2 Hung^{tg/-};p35^{-/-} (SMA;p35^{-/}) mouse strain, and SMA disease phenotypes in these mice were examined. Kinase assay showed that endogenous Cdk5 activity was reduced in SMA mice by p35 knockout (FIG. 11). Further, mitochondrial transport and fragmentation defects as critical SMA disease phenotypes were revealed. Cdk5 has also been implicated in regulating mitochondrial transport and fission. Therefore, whether genetic knockout of p35 in SMA mice could attenuate mitochondrial transport and fragmentation defects was tested. Time-lapse confocal live imaging was used to measure mitochondrial transport and size in primary mouse motor neurons from SMA mice and SMA;p35^{-/-} mice expressing DsRed tagged with mitochondria targeting sequence (mito-DsRed). Motor neurons from SMA mice exhibited a significant decrease in the number of mobile mitochondria, total mitochondrial travel distance, and retrograde transport compared to those from wild type mice (FIGS. 5A-5H). Notably, retrograde transport of mitochondria (FIG. 5H) was particularly affected in SMA motor neurons, consistent with findings that Cdk5 hyperactivation by p25 specifically disrupts dynein-mediated retrograde axonal transport. Importantly, all three mitochondrial transport defects were restored to wild type mouse level in motor neurons from SMA;p35^{-/-} compound mutant mice (FIGS. 5A-5H), indicating that p35 knockout can rescue mitochondria mobility defects in SMA. In addition to mitochondrial transport, mitochondrial size was also examined. Fragmented mitochondria exhibit decreased cristae density leading to impaired ATP synthesis, increased oxidative stress and mitochondrial DNA leakage-induced neuroinflammation, potentially contributing to neurodegeneration. Cdk5 hyperactivation has also been associated with increased mitochondria fission and fragmentation. Indeed, SMA mouse motor neurons showed short and fragmented morphology compared to those from wild type mice, while in SMA;p35^{-/-} mice these defects were rescued to wild type levels (FIGS. 5I, 5J). Collectively, these results show the critical role of Cdk5 hyperactivation by p25 in causing mitochondrial defects in motor neurons affected by SMA.

### Example 6

Genetic Knockout of p35 Alleviates Disease Phenotypes in SMA Mice. To further investigate the role of aberrant Cdk5 activation in SMA pathogenesis in vivo, other SMA disease phenotypes in SMA;p35^{-/-} compound mutant mice were examined. During SMA pathogenesis, motor neurons are characterized by the loss of excitatory synapse innervation on motor neuron cell bodies. Thus, whether genetic knockout of p35 could rescue the excitatory synapse loss phenotype was tested. The number of excitatory synapses on lumber level spinal motor neurons by the colocalization of glutamatergic synapse marker vesicular glutamate transporter 1 (vGluT1) with motor neuron marker choline acetyltransferase (ChAT) (FIGS. 6A-6E) was quantified. The number of excitatory synaptic boutons on motor neurons showed significant reduction in SMA mice, but recovered to wild type level in SMA;p35^{-/-} compound mutant mice (FIGS. 6A-6E), indicating p35 knockout rescues the excitatory synapse loss phenotype in SMA mice.

The reduced excitatory synapse formation on SMA motor neurons may change their functions. So functional properties of motor neurons in wild type, SMA, SMA;p35^{-/-} and p35^{-/-} mice was compared using electrophysiological recordings. Whole cell patch clamp recording of wild type, SMA, SMA;p35^{-/-}, and p35^{-/-} mouse spinal cord tissue slices showed that motor neurons from all four genotypes were undistinguishable in whole cell input resistance, and action potential (AP) characteristics including AP duration, and AP rates of rise and fall (Table 1). However, SMA motor neurons showed hyperexcitability in both their intrinsic currents and their firing behavior. The persistent inward current (PIC), a depolarizing current intrinsic to all neurons that sets the level of neuronal excitability, was significantly larger in SMA motor neurons, and was rescued to wild type levels by genetic knockout of p35^{-/-} in SMA;p35^{-/-} mice (FIG. 6G and Table 1). In addition, motor neuron firing behavior showed similar changes, with SMA motor neurons displaying a hyperpolarized threshold voltage, which was significantly restored in SMA;p35^{-/-} motor neurons (FIG. 6H and FIG). The hyperexcitability of SMA motor neurons observed in this study, including changes in the persistent inward current and action potential threshold voltage, supports this. The finding that hyperexcitability associated with SMA motor neurons could be reduced by genetic knockout of p35 establishes a critical role for Cdk5 aberrant activation in motor neuron defects and SMA pathogenesis.

TABLE 1
Electrophysiological characteristics of spinal motor neurons from wild
type, SMA, SMA; p35^{-/-}, and p35^{-/-} mice.

| | Wild Type | SMA | SMA; p35^{-/-} | p35^{-/-} |
|---|---|---|---|---|
| | Mean ± | Mean ± | Mean ± | Mean ± |
| | SEM (n) | SEM (n) | SEM (n) | SEM (n) |
| RMP (mV) | −59.3 ± | −62.7 ± | −60.7 ± | −58.7 ± |
| | 1.5 (8) | 1.9 (7) | 3.5 (7) | 1.4 (7) |
| Whole cell | 162.9 ± | 170.8 ± | 145.75 ± | 144.3 ± |
| capacitance (pF) | 32.6 (7) | 21.6(7) | 27.5 (7) | 40.1 (7) |
| Whole cell input | 25.4 ± | 34.6 ± | 36.4 ± | 44.6 ± |
| resistance (MΩ) | 2.8 (8) | 2.1 (7) | 2.8 (7) | 11.8 (7) |
| PIC amplitude | 246.1 ± | 425.8 ± | 252.1 ± | 209.8 ± |
| (pA) ** | 59.9 (8) | 39.8 (7) | 26.2 (7) | 20.6 (7) |
| PIC onset | −32.3 ± | −44.5 ± | −42.7 ± | −39.3 ± |
| voltage (mV) | 3.2 (8) | 2.7 (7) | 2.6 (7) | 2.8 (7) |
| PIC peak | −18.0 ± | −18.1 ± | −25.1 ± | −19.5 ± |
| voltage (mV) | 3.1 (8) | 4.1 (7) | 3.0 (7) | 3.3 (7) |
| AP duration | 0.8 ± | 0.8 ± | 0.9 ± | 0.9 ± |
| (ms) | 0.1 (5) | 0.1 (5) | 0.1(6) | 0.1 (5) |
| AP rate of rise | 101.1 ± | 102.2 ± | 100.1 ± | 83.4 ± |
| (mV/ms) | 8.3 (5) | 10.7 (5) | 10.1 (6) | 9.5 (5) |
| AP rate of fall | 80.3 ± | 73.6 ± | 68.4 ± | 60.3 ± |
| (mV/ms) | 9.8 (5) | 7.1 (5) | 6.5 (6) | 4.3 (5) |
| Voltage of | −25.9 ± | −35.0 ± | −29.3 ± | −30.1 ± |
| threshold (mV) * | 2.9 (5) | 1.2 (5) | 1.7 (6) | 1.8 (5) |
| I-ON (pA) | 823.1 ± | 577.2 ± | 580.6 ± | 628.4 ± |
| | 73.1 (5) | 54.5 (5) | 141.4 (7) | 229.8 (6) |
| I-OFF (pA) | 618.9 ± | 430.2 ± | 613.6 ± | 692.7 ± |
| | 72.6 (5) | 39.0 (5) | 186.1 (7) | 268.2 (6) |

* P < 0.05,
** P < 0.01,
One-way ANOVA with Tukey HSD post hoc analysis. n: The number of recorded motor neurons. RMP: resting membrane potential; PIC: persistent inward current; AP: action potential; I-ON: current at which action potentials were elicited; I-OFF: current at which action potentials were ceased.

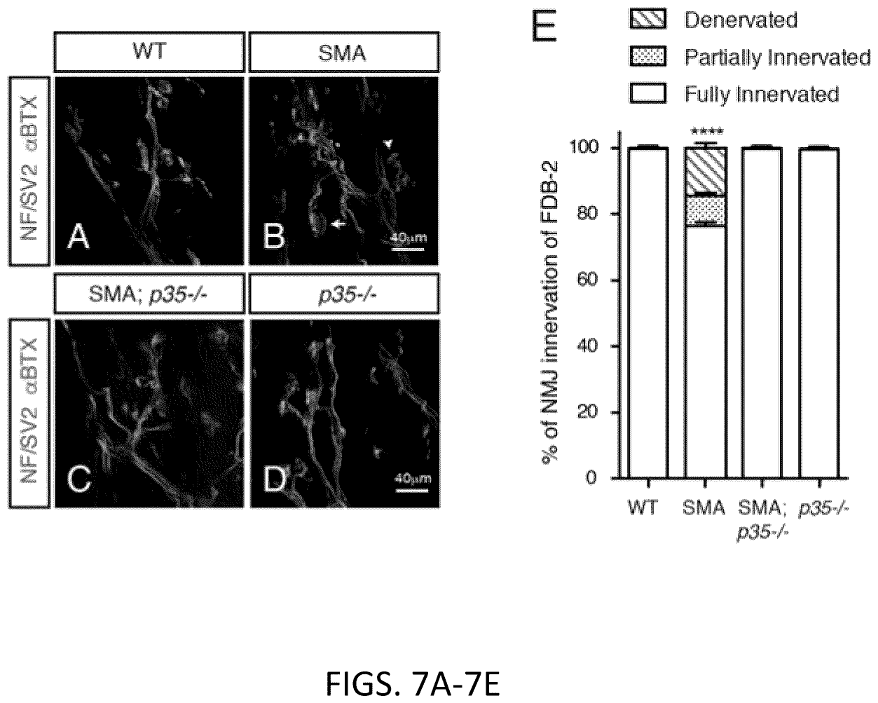

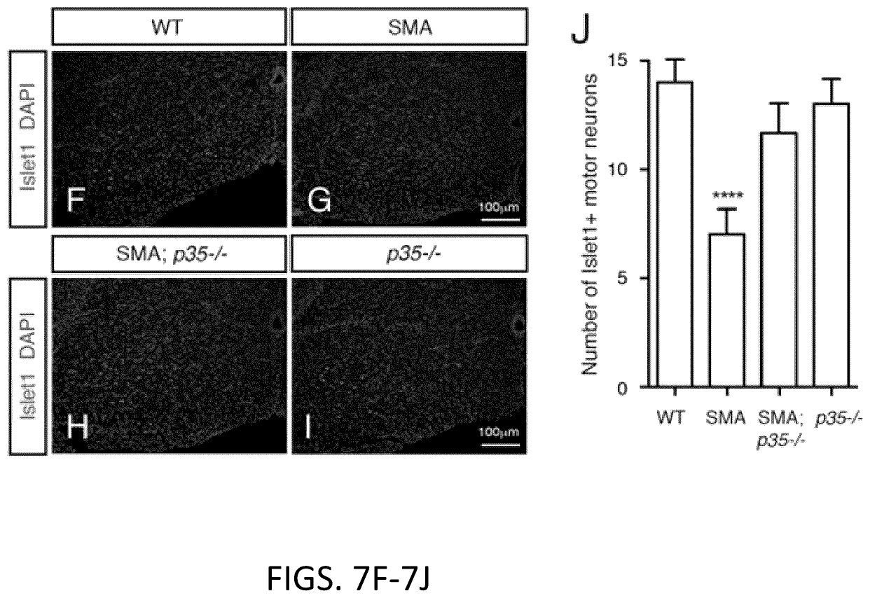

In addition to central excitatory synapse loss, another early and key pathogenic characteristic of SMA disease is compromised innervation of the NMJ by motor axons. In SMA mouse models, NMJ denervation was found in many muscles including the flexor digitorum brevis controlling the second digit (FDB-2). Therefore, NMJ innervation on FDB-2 in wild type, SMA, SMA;p35^{-/-} and p35^{-/-} mice was examined. NMJ was stained with anti-neurofilament and anti-synaptic vesicle 2 (SV2) antibodies to mark presynaptic structures, and α-bungarotoxin (BTX) to label post-synaptic acetylcholine receptors (FIGS. 7A-7D). NMJs on FDB-2 were fully innervated in wild type and p35^{-/-} mice, but showed significantly increased denervation or partial innervation in SMA mice (FIGS. 7B, 7E). Importantly, FDB-2 NMJ innervation in SMA;p35^{-/-} mice was restored back to wild type level, indicating that genetic knockout of p35 rescues NMJ defects in SMA mice (FIGS. 7A-7E).

To examine motor neuron degeneration in SMA mice, lumbar level spinal cord sections were stained with antibodies recognizing motor neuron marker Isl1. SMA mice showed dramatic reduction of motor neurons compared to wild type mice, reflecting motor neuron degeneration in SMA (FIGS. 7F-7J). The number of motor neurons in SMA;p35^{-/-} compound mutant mice was significantly restored, suggesting a beneficial effect of p35 knockout on motor neuron degeneration in SMA pathogenesis (FIGS. 7F, 7H, 7J). The survival life span of SMA;p35^{-/-} double mutant mice was not improved, similar to results in other SMA genetic rescue studies. This is likely because of the defects in cardiovascular and other systems in SMA mice that cannot be rescued by genetic removal of p35, as p35 is predominantly expressed in neurons. Taken together, these results indicate that genetic knockout of p35 reduces motor neuron degeneration in SMA mice.

### Example 7

Inhibition of Cdk5 Signaling Reduces Human SMA iPSC Motor Neuron Degeneration. Attenuating upregulated Cdk5 activity in SMA mice eases motor neuron degeneration. Similar improvements achieved in motor neurons from human SMA patients indicate a therapeutic strategy. SMA or control human iPSC-derived motor neurons were treated with Cdk5 inhibitor BML259 or calpain inhibitor Calpeptin, and examined motor neuron degeneration. Treatment with Cdk5 inhibitor or calpain inhibitor alleviated the degeneration of Isl1-positive motor neurons, while the application of DMSO vehicle as control had no effect (FIGS. 8A-8M). Consistently, inhibition of Cdk5 signaling significantly reduced SMA iPSC motor neuron degeneration measured by staining with a different motor neuron marker choline acetyltransferase (ChAT) or Terminal Transferase dUTP Nick End Labeling (TUNEL) assay (FIGS. 10A, 10B). In addition, pharmacological inhibition of the Cdk5 signaling pathway showed similar beneficial effects on non-integrating SMA iPSC-derived motor neurons (FIG. 9C), indicating that these positive effects are not limited to specific human iPSC lines. Altogether, the findings indicate that inhibiting Cdk5 hyperactivation may represent a new strategy to alleviate SMA motor neuron degeneration in patients.

1. A method of treating or preventing a pediatric neuromuscular disease and/or neurodegenerative disorder comprising administering a Cdk5 inhibitor or calpain inhibitor to a subject suffering from or at risk thereof.

2. (canceled)

3. The method of claim 1, wherein the pediatric neuromuscular disease or the neurodegenerative disorder is spinal muscular atrophy.

4. (canceled)

5. The method of claim 1, wherein the neurodegenerative disorder is a neurodegenerative disorder with dysregulation of Cdk5 signaling and mitochondrial defects.

6-19. (canceled)

20. The method of claim 1, wherein the Cdk5 inhibitor is an inhibitor of Cdk5 activity.

21. The method of claim 20, wherein the Cdk5 inhibitor comprises a BML-259, a CDK5-IN-4, a 25-106, a seliciclib, a roscovitine, a CY-202, a quinazolinone derivative, a quercetin, a dinaciclib, CDK5 inhibitory peptide (CIP), a derivative of a P5 peptide that is conjugated with a cell-penetrating transactivator of transcription (TAT) sequence and a FITC tag, a statin, a metformin, a fenofibrate, and a rosiglitazone.

22. The method of claim 1, wherein the Cdk5 inhibitor is an inhibitor of Cdk5 expression.

23. The method of claim 22, wherein the inhibitor of Cdk5 expression is an shRNA, a miRNA, a morpholino, a ribozyme, an antisense nucleic acid molecule, or a CRISPR-based construct.

24. The method of claim 1, wherein the calpain inhibitor is an inhibitor of calpain activity.

25. The method of claim 24, wherein calpain inhibitor comprises calpeptin, N-acetyl-leu-leu-norleucinal (ALLN), PD150606, SNJ-1945, AK275, MDL28170, Gabadur, Neurodur, E-64-d, SCI Leupeptin, and NA-184.

26. The method of claim 1, wherein the calpain inhibitor is an inhibitor of calpain expression.

27. The method of claim 26, wherein the inhibitor of calpain expression is an shRNA, a miRNA, a morpholino, a ribozyme, an antisense nucleic acid molecule, or a CRISPR-based construct.

28. The method of claim 1, comprising co-administration of the Cdk5 inhibitor and an additional therapeutic.

29. The method of claim 28, wherein the additional therapeutic is a calpain inhibitor.

30. The method of claim 1, comprising co-administration of the calpain inhibitor and an additional therapeutic.

31. The method of claim 1, wherein administering comprises co-administration of metformin and fenofibrate, co-administration of metformin and rosiglitazone, or co-administration of rosiglitazone and glimepiride.

32. The method of claim 1 wherein the Cdk5 inhibitor comprises a formulation comprising a tablet, a capsule, and an infusion.

33. The method of claim 1 wherein administering comprises oral, parenteral, intramuscular, intraperitoneal, intravenous, intracerebroventricular, intracisternal, intratracheal, intranasal, subcutaneous, via injection or infusion, via inhalation, spray, nasal, vaginal, rectal, sublingual, and topical administration.
